(19) United States
(12) Patent Application Publication
(10) Pub. No.: US 2025/0195820 A1
(43) Pub. Date: Jun. 19, 2025

(54) HEAT AND MOISTURE EXCHANGERS

(71) Applicant: RESMED PTY LTD, Bella Vista (AU)

(72) Inventor: Muditha Pradeep DANTANARAYANA, Sydney (AU)

(21) Appl. No.: 18/985,935

(22) Filed: Dec. 18, 2024

(30) Foreign Application Priority Data
Dec. 19, 2023 (AU) 2023904128

(51) Int. Cl. A61M 16/10; F28D 21/00

(52) U.S. Cl. CPC A61M 16/1045; F28D 21/0015; F28D 2021/005

(57) ABSTRACT

A heat and moisture exchanger (HME) for heating and/or humidifying the flow of air for breathing by a patient using heat and/or moisture recovered from a flow of gases exhaled by the patient, the HME having a heat and moisture exchange component and a thermally conductive member to facilitate heat transfer within the HME, the thermally conductive member being in contact with, or incorporated within, the heat and moisture exchange component, for incorporation into a patient interface having a plenum chamber pressurisable to a therapeutic pressure of at least 6 cm H2O above ambient air pressure. The HME is configured to be removably connected directly or indirectly to the plenum chamber.

### CROSS-REFERENCE TO RELATED APPLICATIONS

This application claims priority to Australian Provisional Patent Application No. 2023904128 filed Dec. 19, 2023, the entire contents of which are hereby incorporated by reference.

### 1. BACKGROUND OF THE TECHNOLOGY 1.1 Field of the Technology

The present technology relates to one or more of the screening, diagnosis, monitoring, treatment, prevention and amelioration of respiratory-related disorders. The present technology also relates to medical devices or apparatus, and their use.

### 1.2 Description of the Related Art

### 1.2.1 Human Respiratory System and its Disorders

The respiratory system of the body facilitates gas exchange. The nose and mouth form the entrance to the airways of a patient.

The airways include a series of branching tubes, which become narrower, shorter and more numerous as they penetrate deeper into the lung. The prime function of the lung is gas exchange, allowing oxygen to move from the inhaled air into the venous blood and carbon dioxide to move in the opposite direction. The trachea divides into right and left main bronchi, which further divide eventually into terminal bronchioles. The bronchi make up the conducting airways, and do not take part in gas exchange. Further divisions of the airways lead to the respiratory bronchioles, and eventually to the alveoli. The alveolated region of the lung is where the gas exchange takes place, and is referred to as the respiratory zone. See “Respiratory Physiology”, by John B. West, Lippincott Williams & Wilkins, 9th edition published 2012.

A range of respiratory disorders exist. Certain disorders may be characterised by particular events, e.g. apneas, hypopneas, and hyperpneas.

Examples of respiratory disorders include Obstructive Sleep Apnea (OSA), Cheyne-Stokes Respiration (CSR), respiratory insufficiency, Obesity Hypoventilation Syndrome (OHS), Chronic Obstructive Pulmonary Disease (COPD), Neuromuscular Disease (NMD) and Chest wall disorders.

Obstructive Sleep Apnea (OSA), a form of Sleep Disordered Breathing (SDB), is characterised by events including occlusion or obstruction of the upper air passage during sleep. It results from a combination of an abnormally small upper airway and the normal loss of muscle tone in the region of the tongue, soft palate and posterior oropharyngeal wall during sleep. The condition causes the affected patient to stop breathing for periods typically of 30 to 120 seconds in duration, sometimes 200 to 300 times per night. It often causes excessive daytime somnolence, and it may cause cardiovascular disease and brain damage. The syndrome is a common disorder, particularly in middle aged overweight males, although a person affected may have no awareness of the problem, e.g. see U.S. Pat. No. 4,944,310 (Sullivan).

Cheyne-Stokes Respiration (CSR) is another form of sleep disordered breathing. CSR is a disorder of a patient's respiratory controller in which there are rhythmic alternating periods of waxing and waning ventilation known as CSR cycles. CSR is characterised by repetitive de-oxygenation and re-oxygenation of the arterial blood. It is possible that CSR is harmful because of the repetitive hypoxia. In some patients CSR is associated with repetitive arousal from sleep, which causes severe sleep disruption, increased sympathetic activity, and increased afterload, e.g. see U.S. Pat. No. 6,532,959 (Berthon-Jones).

Respiratory failure is an umbrella term for respiratory disorders in which the lungs are unable to inspire sufficient oxygen or exhale sufficient CO2 to meet the patient's needs. Respiratory failure may encompass some or all of the following disorders.

A patient with respiratory insufficiency (a form of respiratory failure) may experience abnormal shortness of breath on exercise.

Obesity Hypoventilation Syndrome (OHS) is defined as the combination of severe obesity and awake chronic hypercapnia, in the absence of other known causes for hypoventilation. Symptoms include dyspnea, morning headache and excessive daytime sleepiness.

Chronic Obstructive Pulmonary Disease (COPD) encompasses any of a group of lower airway diseases that have certain characteristics in common. These include increased resistance to air movement, extended expiratory phase of respiration, and loss of the normal elasticity of the lung. Examples of COPD are emphysema and chronic bronchitis. COPD is caused by chronic tobacco smoking (primary risk factor), occupational exposures, air pollution and genetic factors. Symptoms include: dyspnea on exertion, chronic cough and sputum production.

Neuromuscular Disease (NMD) is a broad term that encompasses many diseases and ailments that impair the functioning of the muscles either directly via intrinsic muscle pathology, or indirectly via nerve pathology. Some NMD patients are characterised by progressive muscular impairment leading to loss of ambulation, being wheelchair-bound, swallowing difficulties, respiratory muscle weakness and, eventually, death from respiratory failure. Neuromuscular disorders can be divided into rapidly progressive and slowly progressive: (i) Rapidly progressive disorders: Characterised by muscle impairment that worsens over months and results in death within a few years (e.g. Amyotrophic lateral sclerosis (ALS) and Duchenne muscular dystrophy (DMD) in teenagers); (ii) Variable or slowly progressive disorders: Characterised by muscle impairment that worsens over years and only mildly reduces life expectancy (e.g. Limb girdle, Facioscapulohumeral and Myotonic muscular dystrophy). Symptoms of respiratory failure in NMD include: increasing generalised weakness, dysphagia, dyspnea on exertion and at rest, fatigue, sleepiness, morning headache, and difficulties with concentration and mood changes.

Chest wall disorders are a group of thoracic deformities that result in inefficient coupling between the respiratory muscles and the thoracic cage. The disorders are usually characterised by a restrictive defect and share the potential of long term hypercapnic respiratory failure. Scoliosis and/or kyphoscoliosis may cause severe respiratory failure. Symptoms of respiratory failure include: dyspnea on exertion, peripheral oedema, orthopnea, repeated chest infections, morning headaches, fatigue, poor sleep quality and loss of appetite.

A range of therapies have been used to treat or ameliorate such conditions. Furthermore, otherwise healthy individuals may take advantage of such therapies to prevent respiratory disorders from arising. However, these have a number of shortcomings.

### 1.2.2 Therapies

Various respiratory therapies, such as Continuous Positive Airway Pressure (CPAP) therapy, Non-invasive ventilation (NIV), Invasive ventilation (IV), and High Flow Therapy (HFT) have been used to treat one or more of the above respiratory disorders.

### 1.2.3 Respiratory Pressure Therapies

Respiratory pressure therapy is the application of a supply of air to an entrance to the airways at a controlled target pressure that is nominally positive with respect to atmosphere throughout the patient's breathing cycle (in contrast to negative pressure therapies such as the tank ventilator or cuirass).

Continuous Positive Airway Pressure (CPAP) therapy has been used to treat Obstructive Sleep Apnea (OSA). The mechanism of action is that continuous positive airway pressure acts as a pneumatic splint and may prevent upper airway occlusion, such as by pushing the soft palate and tongue forward and away from the posterior oropharyngeal wall. Treatment of OSA by CPAP therapy may be voluntary, and hence patients may elect not to comply with therapy if they find devices used to provide such therapy one or more of: uncomfortable, difficult to use, expensive and aesthetically unappealing.

Non-invasive ventilation (NIV) provides ventilatory support to a patient through the upper airways to assist the patient breathing and/or maintain adequate oxygen levels in the body by doing some or all of the work of breathing. The ventilatory support is provided via a non-invasive patient interface. NIV has been used to treat CSR and respiratory failure, in forms such as OHS, COPD, NMD and Chest Wall disorders. In some forms, the comfort and effectiveness of these therapies may be improved.

Invasive ventilation (IV) provides ventilatory support to patients that are no longer able to effectively breathe themselves and may be provided using a tracheostomy tube or endotracheal tube. In some forms, the comfort and effectiveness of these therapies may be improved.

### 1.2.3.1 Flow Therapies

Not all respiratory therapies aim to deliver a prescribed therapeutic pressure. Some respiratory therapies aim to deliver a prescribed respiratory volume, by delivering an inspiratory flow rate profile over a targeted duration, possibly superimposed on a positive baseline pressure. In other cases, the interface to the patient's airways is ‘open’ (unsealed) and the respiratory therapy may only supplement the patient's own spontaneous breathing with a flow of conditioned or enriched gas. In one example, High Flow therapy (HFT) is the provision of a continuous, heated, humidified flow of air to an entrance to the airway through an unsealed or open patient interface at a “treatment flow rate” that may be held approximately constant throughout the respiratory cycle. The treatment flow rate is nominally set to exceed the patient's peak inspiratory flow rate. HFT has been used to treat OSA, CSR, respiratory failure, COPD, and other respiratory disorders. One mechanism of action is that the high flow rate of air at the airway entrance improves ventilation efficiency by flushing, or washing out, expired CO2 from the patient's anatomical deadspace. Hence, HFT is thus sometimes referred to as a deadspace therapy (DST). Other benefits may include the elevated warmth and humidification (possibly of benefit in secretion management) and the potential for modest elevation of airway pressures. As an alternative to constant flow rate, the treatment flow rate may follow a profile that varies over the respiratory cycle.

Another form of flow therapy is long-term oxygen therapy (LTOT) or supplemental oxygen therapy. Doctors may prescribe a continuous flow of oxygen enriched air at a specified oxygen concentration (from 21%, the oxygen fraction in ambient air, to 100%) at a specified flow rate (e.g., 1 litre per minute (LPM), 2 LPM, 3 LPM, etc.) to be delivered to the patient's airway.

### 1.2.4 Respiratory Therapy Systems

These respiratory therapies may be provided by a respiratory therapy system or device. Such systems and devices may also be used to screen, diagnose, or monitor a condition without treating it.

A respiratory therapy system may comprise a Respiratory Pressure Therapy Device (RPT device), an air circuit, a humidifier, a patient interface, an oxygen source, and data management.

### 1.2.4.1 Patient Interface

A patient interface may be used to interface respiratory equipment to its wearer, for example by providing a flow of air to an entrance to the airways. The flow of air may be provided via a mask to the nose and/or mouth, a tube to the mouth or a tracheostomy tube to the trachea of a patient. Depending upon the therapy to be applied, the patient interface may form a seal, e.g., with a region of the patient's face, to facilitate the delivery of gas at a pressure at sufficient variance with ambient pressure to effect therapy, e.g., at a positive pressure of about 10 cmH2O relative to ambient pressure. For other forms of therapy, such as the delivery of oxygen, the patient interface may not include a seal sufficient to facilitate delivery to the airways of a supply of gas at a positive pressure of about 10 cmH2O. For flow therapies such as nasal HFT, the patient interface is configured to insufflate the nares but specifically to avoid a complete seal. One example of such a patient interface is a nasal cannula.

Certain mask systems may be functionally unsuitable for the present field. For example, purely ornamental masks may be unable to maintain a suitable pressure. Mask systems used for underwater swimming or diving may be configured to guard against ingress of water from an external higher pressure, but not to maintain air internally at a higher pressure than ambient.

Certain masks may be clinically unfavourable for the present technology e.g. if they block airflow via the nose and only allow it via the mouth.

Certain masks may be uncomfortable or impractical for the present technology if they require a patient to insert a portion of a mask structure in their mouth to create and maintain a seal via their lips.

Certain masks may be impractical for use while sleeping, e.g. for sleeping while lying on one's side in bed with a head on a pillow.

Certain masks may cause some patients a feeling of claustrophobia, unease and/or may feel overly obtrusive.

The design of a patient interface presents a number of challenges. The face has a complex three-dimensional shape. The size and shape of noses and heads varies considerably between individuals. Since the head includes bone, cartilage and soft tissue, different regions of the face respond differently to mechanical forces. The jaw or mandible may move relative to other bones of the skull. The whole head may move during the course of a period of respiratory therapy.

Consequently, some masks suffer from being obtrusive, aesthetically undesirable, costly, poorly fitting, difficult to use, and/or uncomfortable especially when worn for long or when a patient is unfamiliar with a system. Wrongly sized masks can give rise to reduced compliance, reduced comfort and poorer patient outcomes. Masks designed solely for aviators, masks designed as part of personal protection equipment (e.g. filter masks), SCUBA masks, or for the administration of anaesthetics may be tolerable for their original application, but nevertheless such masks may be undesirably uncomfortable to be worn for extended periods of time, e.g., several hours. This discomfort may lead to a reduction in patient compliance with therapy, especially if the mask is to be worn during sleep.

CPAP therapy is highly effective to treat certain respiratory disorders, provided patients comply with therapy. If a mask is uncomfortable, or difficult to use a patient may not comply with therapy. Since it is often recommended that a patient regularly wash their mask, if a mask is difficult to clean (e.g., difficult to assemble or disassemble), patients may not clean their mask and this may impact on patient compliance.

While a mask for other applications (e.g. aviators) may not be suitable for use in treating sleep disordered breathing, a mask designed for use in treating sleep disordered breathing may be suitable for other applications.

For these reasons, patient interfaces for delivery of CPAP during sleep form a distinct field.

### 1.2.4.1.1 Seal-Forming Structure

Patient interfaces may include a seal-forming structure. Since it is in direct contact with the patient's face, the shape and configuration of the seal-forming structure can have a direct impact the effectiveness and comfort of the patient interface.

A patient interface may be partly characterised according to the design intent of where the seal-forming structure is to engage with the face in use. In one form of patient interface, a seal-forming structure may comprise a first sub-portion to form a seal around the left naris and a second sub-portion to form a seal around the right naris. In one form of patient interface, a seal-forming structure may comprise a single element that surrounds both nares in use. Such single element may be designed to for example overlay an upper lip region and a nasal bridge region of a face. In one form of patient interface a seal-forming structure may comprise an element that surrounds a mouth region in use, e.g. by forming a seal on a lower lip region of a face. In one form of patient interface, a seal-forming structure may comprise a single element that surrounds both nares and a mouth region in use. These different types of patient interfaces may be known by a variety of names by their manufacturer including nasal masks, full-face masks, nasal pillows, nasal puffs and oro-nasal masks.

A seal-forming structure that may be effective in one region of a patient's face may be inappropriate in another region, e.g. because of the different shape, structure, variability and sensitivity regions of the patient's face. For example, a seal on swimming goggles that overlays a patient's forehead may not be appropriate to use on a patient's nose.

Certain seal-forming structures may be designed for mass manufacture such that one design is able to fit and be comfortable and effective for a wide range of different face shapes and sizes. To the extent to which there is a mismatch between the shape of the patient's face, and the seal-forming structure of the mass-manufactured patient interface, one or both must adapt in order for a seal to form.

One type of seal-forming structure extends around the periphery of the patient interface, and is intended to seal against the patient's face when force is applied to the patient interface with the seal-forming structure in confronting engagement with the patient's face. The seal-forming structure may include an air or fluid filled cushion, or a moulded or formed surface of a resilient seal element made of an elastomer such as a rubber. With this type of seal-forming structure, if the fit is not adequate, there will be gaps between the seal-forming structure and the face, and additional force will be required to force the patient interface against the face in order to achieve a seal.

Another type of seal-forming structure incorporates a flap seal of thin material positioned about the periphery of the mask so as to provide a self-sealing action against the face of the patient when positive pressure is applied within the mask. Like the previous style of seal forming portion, if the match between the face and the mask is not good, additional force may be required to achieve a seal, or the mask may leak. Furthermore, if the shape of the seal-forming structure does not match that of the patient, it may crease or buckle in use, giving rise to leaks.

Another type of seal-forming structure may comprise a friction-fit element, e.g. for insertion into a naris, however some patients find these uncomfortable.

Another form of seal-forming structure may use adhesive to achieve a seal. Some patients may find it inconvenient to constantly apply and remove an adhesive to their face.

A range of patient interface seal-forming structure technologies are disclosed in the following patent applications: WO 1998/004310; WO 2006/074513; WO 2010/135785.

One form of nasal pillow is found in the Adam Circuit manufactured by Puritan Bennett. Another nasal pillow, or nasal puff is the subject of U.S. Pat. No. 4,782,832 (Trimble et al.), assigned to Puritan-Bennett Corporation.

ResMed Inc. has manufactured the following products that incorporate nasal pillows: SWIFT™ nasal pillows mask, SWIFT™ II nasal pillows mask, SWIFT™ LT nasal pillows mask, SWIFT™ FX nasal pillows mask and MIRAGE LIBERTY™ full-face mask. The following patent applications describe examples of nasal pillows masks: International Patent Application WO 2004/073778 (describing amongst other things aspects of the SWIFT™ nasal pillows mask), US Patent Application 2009/0044808 (describing amongst other things aspects of the SWIFT™ LT nasal pillows mask); International Patent Applications WO 2005/063328 and WO 2006/130903 (describing amongst other things aspects of the MIRAGE LIBERTY™ full-face mask); International Patent Application WO 2009/052560 (describing amongst other things aspects of the SWIFT™ FX nasal pillows).

### 1.2.4.1.2 Positioning and Stabilising Structure

A seal-forming structure of a patient interface used for positive air pressure therapy is subject to the corresponding force of the air pressure to disrupt a seal. Thus a variety of techniques have been used to position the seal-forming structure, and to maintain it in sealing relation with the appropriate portion of the face. Several factors may be considered when comparing different positioning and stabilising techniques. These include: how effective the technique is at maintaining the seal-forming structure in the desired position and in sealed engagement with the face during use of the patient interface; how comfortable the interface is for the patient; whether the patient feels intrusiveness and/or claustrophobia when wearing the patient interface; and aesthetic appeal.

Another technique is the use of one or more straps and/or stabilising harnesses. Many such harnesses suffer from being one or more of ill-fitting, bulky, uncomfortable and awkward to use.

### 1.2.4.2 Respiratory Pressure Therapy (RPT) Device

A respiratory pressure therapy (RPT) device may be used individually or as part of a system to deliver one or more of a number of therapies described above, such as by operating the device to generate a flow of air for delivery to an interface to the airways. The flow of air may be pressure-controlled (for respiratory pressure therapies) or flow-controlled (for flow therapies such as HFT). Thus RPT devices may also act as flow therapy devices. Examples of RPT devices include a CPAP device and a ventilator.

The designer of a device may be presented with an infinite number of choices to make. Design criteria often conflict, meaning that certain design choices are far from routine or inevitable. Furthermore, the comfort and efficacy of certain aspects may be highly sensitive to small, subtle changes in one or more parameters.

### 1.2.4.3 Air Circuit

An air circuit is a conduit or a tube constructed and arranged to allow, in use, a flow of air to travel between two components of a respiratory therapy system such as the RPT device and the patient interface. In some cases, there may be separate limbs of the air circuit for inhalation and exhalation. In other cases, a single limb air circuit is used for both inhalation and exhalation.

### 1.2.4.4 Humidifier

Delivery of a flow of air without humidification may cause drying of airways. The use of a humidifier with an RPT device and the patient interface produces humidified gas that minimizes drying of the nasal mucosa and increases patient airway comfort. In addition, in cooler climates, warm air applied generally to the face area in and about the patient interface is more comfortable than cold air.

A range of artificial humidification devices and systems are known, however they may not fulfil the specialised requirements of a medical humidifier.

Medical humidifiers are used to increase humidity and/or temperature of the flow of air in relation to ambient air when required, typically where the patient may be asleep or resting (e.g. at a hospital). A medical humidifier for bedside placement may be small. A medical humidifier may be configured to only humidify and/or heat the flow of air delivered to the patient without humidifying and/or heating the patient's surroundings. Room-based systems (e.g. a sauna, an air conditioner, or an evaporative cooler), for example, may also humidify air that is breathed in by the patient, however those systems would also humidify and/or heat the entire room, which may cause discomfort to the occupants. Furthermore, medical humidifiers may have more stringent safety constraints than industrial humidifiers

While a number of medical humidifiers are known, they can suffer from one or more shortcomings. Some medical humidifiers may provide inadequate humidification, some are difficult or inconvenient to use by patients.

### 1.2.4.5 Heat and Moisture Exchanger (HME)

Heat and moisture exchangers are generally made up of foam, paper, or an alternative substance capable of acting as a condensation and absorption surface. The material may carry hygroscopic salts to improve the water-retaining capacity. Suitable salts include calcium chloride.

HMEs may be utilized in RPT therapy, such as in PAP therapy, to partially recover heat and moisture present in exhaled gas from a patient's airways. This heat and moisture can be retained and recycled to the patient in a passive manner as a flow of breathable gas passes through the HME prior to inspiration. Thus, the use of HMEs can provide at least some of the needed moisture and humidity (generally recognized as >10 mg/l) to many patients during PAP therapy to minimize any detrimental effects associated with PAP therapy with non-humidified ambient air whilst avoiding the need for a heated humidifier system, or at least reducing the load on such a humidifier system if one is present. The use of a HME may also lower the possibility of occlusion caused by condensation in air delivery tubes.

The use of a HME in PAP therapy can avoid or reduce the need for additional power required with heated humidifiers and may reduce the need for extra associated components. This may reduce manufacturing costs and may also reduce the overall size of the CPAP therapy unit.

A problem common with the use of HMEs in CPAP therapy relates to the ability of the HME to provide sufficient heat and moisture while also minimizing flow impedance and maintaining comfortable and safe levels of CO2 washout. Flow impedance may affect patient breathing effort (work of breathing) and may also impact event (apnoea, hypopnoea, snore) detection algorithms, so in many cases it is sought to be minimized. Furthermore, consideration should also be given to minimising heat and moisture loss from venting.

Because HMEs typically create an impedance to flow from the patient's airways to the vent, patient interfaces which use HMEs may suffer from reduced CO2 washout. This effect may be exacerbated if there is a large volume between the patient's airways and the HME.

Moreover, many current designs of HMEs do not allow for sufficient moisture exchange during patient exhalation to provide sufficient humidification levels to the patient. Thus, there is a need to provide superior configurations and designs for HME use in RPT therapy, such as PAP therapy, to achieve desired patient humidification whilst having acceptable impedance and CO2 washout. It is also desirable that an HME for use with a flexible patient interface does not adversely affect the ability of the patient interface to deform in desirable ways.

Over time, HMEs positioned within a patient interface may reach a steady-state temperature, reducing the ability of the HME to retain moisture and subsequently effectively humidify air passing through the HME.

### 1.2.4.6 Vent Technologies

Some forms of treatment systems may include a vent to allow the washout of exhaled carbon dioxide. The vent may allow a flow of gas from an interior space of a patient interface, e.g., the plenum chamber, to an exterior of the patient interface, e.g., to ambient.

The vent may comprise an orifice and gas may flow through the orifice in use of the mask. Many such vents are noisy. Others may become blocked in use and thus provide insufficient washout. Some vents may be disruptive of the sleep of a bed partner 1100 of the patient 1000, e.g. through noise or focused airflow.

ResMed Inc. has developed a number of improved mask vent technologies, e.g. see International Patent Application Publication No. WO 1998/034665; International Patent Application Publication No. WO 2000/078381; U.S. Pat. No. 6,581,594; US Patent Application Publication No. US 2009/0050156; US Patent Application Publication No. 2009/0044808.

Table of noise of prior masks (ISO 17510-2:2007, 10 cmH2O pressure at 1 m)

| | | | A-weighted | |
|---|---|---|---|---|
| | | A-weighted | sound | |
| | | sound power | pressure | |
| | | level dB(A) | dB(A) | Year |
| Mask name | Mask type | (uncertainty) | (uncertainty) | (approx.) |
| Glue-on (*) | nasal | 50.9 | 42.9 | 1981 |
| ResCare | nasal | 31.5 | 23.5 | 1993 |
| standard (*) | | | | |
| ResMed | nasal | 29.5 | 21.5 | 1998 |
| MirageTM (*) | | | | |
| ResMed | nasal | 36 (3) | 28 (3) | 2000 |
| UltraMirageTM | | | | |
| ResMed | nasal | 32 (3) | 24 (3) | 2002 |
| Mirage | | | | |
| ActivaTM | | | | |
| ResMed | nasal | 30 (3) | 22 (3) | 2008 |
| Mirage | | | | |
| MicroTM | | | | |
| ResMed | nasal | 29 (3) | 22 (3) | 2008 |
| MirageTM | | | | |
| SoftGel | | | | |
| ResMed | nasal | 26 (3) | 18 (3) | 2010 |
| MirageTM FX | | | | |
| ResMed | nasal pillows | 37 | 29 | 2004 |
| Mirage | | | | |
| SwiftTM (*) | | | | |
| ResMed | nasal pillows | 28 (3) | 20 (3) | 2005 |
| Mirage | | | | |
| SwiftTM II | | | | |
| ResMed | nasal pillows | 25 (3) | 17 (3) | 2008 |
| Mirage | | | | |
| SwiftTM LT | | | | |
| ResMed AirFit | nasal pillows | 21 (3) | 13 (3) | 2014 |
| P10 | | | | |

(*) one specimen only, measured using test method specified in ISO 3744 in CPAP mode at 10 cmH2O)

Sound pressure values of a variety of objects are listed below

| | A-weighted sound | |
|---|---|---|
| Object | pressure dB(A) | Notes |
| Vacuum cleaner: Nilfisk | 68 | ISO 3744 at |
| Walter Broadly Litter Hog: | | 1 m distance |
| B+ Grade | | |
| Conversational speech | 60 | 1 m distance |
| Average home | 50 | |
| Quiet library | 40 | |
| Quiet bedroom at night | 30 | |
| Background in TV studio | 20 | |

### 2 BRIEF SUMMARY OF THE TECHNOLOGY

The present technology is directed towards providing medical devices used in the screening, diagnosis, monitoring, amelioration, treatment, or prevention of respiratory disorders having one or more of improved comfort, cost, efficacy, ease of use and manufacturability.

A first aspect of the present technology relates to apparatus used in the screening, diagnosis, monitoring, amelioration, treatment or prevention of a respiratory disorder.

Another aspect of the present technology relates to methods used in the screening, diagnosis, monitoring, amelioration, treatment or prevention of a respiratory disorder.

An aspect of certain forms of the present technology is to provide methods and/or apparatus that improve the compliance of patients with respiratory therapy.

One form of the present technology comprises a patient interface for providing a flow of air at therapeutic pressure for breathing by a patient, the patient interface including a heat and moisture exchanger (HME) allowing heat and moisture capture from air exhaled by the patient, and/or humidification of air inhaled by the patient.

In examples, the heat and moisture exchanger (HME) comprises a thermally conductive member and a heat and moisture exchange component.

In a further form of the present technology, the patient interface comprises:
a plenum chamber pressurisable to a therapeutic pressure of at least 6 cm H2O above ambient air pressure, said plenum chamber including a plenum chamber inlet port sized and structured to receive a flow of air at the therapeutic pressure for breathing by a patient,
a seal-forming structure constructed and arranged to form a seal with a region of the patient's face surrounding an entrance to the patient's airways, said seal-forming structure having a hole therein such that the flow of air at said therapeutic pressure is delivered to at least an entrance to the patient's nares, the seal-forming structure constructed and arranged to maintain said therapeutic pressure in the plenum chamber throughout the patient's respiratory cycle in use; and
a vent structure to allow a continuous flow of gases exhaled by the patient from an interior of the plenum chamber to ambient, said vent structure being sized and shaped to maintain the therapeutic pressure in the plenum chamber in use;
   wherein the patient interface comprises a heat and moisture exchanger (HME) for heating and/or humidifying the flow of air for breathing by the patient using heat and/or moisture recovered from the flow of gases exhaled by the patient, the HME comprising a heat and moisture exchange component and a thermally conductive member to facilitate heat transfer within the HME, the thermally conductive member in contact with, or incorporated within, the heat and moisture exchange component.

In further examples, the HME comprises a heat and moisture exchange component and a thermally conductive element to increase heat transfer within the HME, the thermally conductive element in contact with, or incorporated within, the heat and moisture exchange component.

In examples:
a) the HME is located in the plenum chamber of the patient interface and the thermally conductive member or portion thereof is located on the inside of the plenum chamber between the patient's face and the heat and moisture exchange component;
b) the patient interface includes a tube connected to the plenum chamber inlet port, the HME connected to the tube at an opposing tube end distal from the plenum chamber and optionally, the thermally conductive member of the HME is located between the plenum chamber and the heat and moisture exchange component of the HME;
c) the thermally conductive member is in the form of one or more of; a metal sheet, a foil sheet, mesh, perforated metal sheeting, wire or plate;
d) the thermally conductive member is selected from silver, copper, gold, steel, aluminium nitride, silicon carbide, aluminium, tungsten, graphite or zinc;
e) the thermal conductivity of the thermally conductive member is between 10 W/m·K-1000 W/m·K.;
f) the thermal conductivity of the thermally conductive member is between 50 and 500 W/m·K;
g) the HME comprises a covering structure, flange, surround, casing or retaining structure formed from, partially formed from, or comprising the thermally conductive member;
h) the thermally conductive member is a covering structure shaped to at least partially receive or be received by at least a portion of the heat and moisture exchange component;
i) the heat and moisture exchange component is impregnated and/or coated with the thermally conductive member;
j) the HME is shaped such that at least a portion of the thermally conductive member is exposed to ambient air;
k) the thermally conductive member is in thermal connection with the ambient air and the heat and moisture exchange component;
l) the HME is removably connected to the patient interface;
m) the heat and moisture exchange component of the HME is removably connected to the thermally conductive member and/or a component of the patient interface;
n) the thermally conductive member of the HME is removably connected to the heat and moisture exchange component and/or a component of the patient interface; and/or
o) the HME comprises a projection structure providing thermal connection between the thermally conductive member and ambient air.

Another form of the technology comprises a heat and moisture exchanger (HME) adapted for use in a patient interface, the HME comprising a heat and moisture exchange component and a thermally conductive member, the thermally conductive member in contact with or incorporated within the heat and moisture exchange component.

A still further form of the present technology comprises a heat and moisture exchanger (HME) for heating and/or humidifying the flow of air for breathing by a patient using heat and/or moisture recovered from a flow of gases exhaled by the patient, the HME comprising a heat and moisture exchange component and a thermally conductive member to facilitate heat transfer within the HME, the thermally conductive member in contact with, or incorporated within, the heat and moisture exchange component, for incorporation into a patient interface, the patient interface comprising;
a plenum chamber pressurisable to a therapeutic pressure of at least 6 cm H2O above ambient air pressure, said plenum chamber including a plenum chamber inlet port sized and structured to receive a flow of air at the therapeutic pressure for breathing by a patient; and
a seal-forming structure constructed and arranged to form a seal with a region of the patient's face surrounding an entrance to the patient's airways, said seal-forming structure having a hole therein such that the flow of air at said therapeutic pressure is delivered to at least an entrance to the patient's nares, the seal-forming structure constructed and arranged to maintain said therapeutic pressure in the plenum chamber throughout the patient's respiratory cycle in use; and
a vent structure to allow a continuous flow of gases exhaled by the patient from an interior of the plenum chamber to ambient, said vent structure being sized and shaped to maintain the therapeutic pressure in the plenum chamber in use; and wherein the HME is connected to the plenum chamber; or
   wherein the patient interface includes a tube connected to the plenum chamber inlet port, the HME connected to the tube at an opposing tube end distal from the plenum chamber.

In examples;
a) the thermally conductive member of the HME or portion thereof is located on or in the plenum chamber between the patient's face and the heat and moisture exchange component of the HME;
b) the thermally conductive member of the HME is located between the plenum chamber and the heat and moisture exchange component of the HME;
c) the thermally conductive member is in the form of one or more of; a metal sheet, a foil sheet, mesh, perforated metal sheeting, wire or plate;
d) the thermally conductive member is selected from silver, copper, gold, steel, aluminium nitride, silicon carbide, aluminium, tungsten, graphite or zinc;
e) the thermal conductivity of the thermally conductive member is between 10 W/m·K-1000 W/m·K.;
f) the thermal conductivity of the thermally conductive member is between 50 and 500 W/m·K;
g) the HME comprises a covering structure, flange, surround, casing or retaining structure formed from, partially formed from, or comprising the thermally conductive member;
h) the thermally conductive member is a covering structure shaped to at least partially receive or be received by at least a portion of the heat and moisture exchange component;
i) the heat and moisture exchange component is impregnated and/or coated with the thermally conductive member;
j) the HME is shaped such that at least a portion of the thermally conductive member is exposed to ambient air;
k) the thermally conductive member is in thermal connection with the ambient air and the heat and moisture exchange component;
l) the HME is removably connected to the patient interface;
m) the heat and moisture exchange component of the HME is removably connected to the thermally conductive member and/or a component of the patient interface;
n) the thermally conductive member of the HME is removably connected to the heat and moisture exchange component and/or a component of the patient interface; and/or
o) the HME comprises a projection structure providing thermal connection between the thermally conductive member and ambient air.

Another aspect of one form of the present technology is a patient interface that is moulded or otherwise constructed with a perimeter shape which is complementary to that of an intended wearer.

An aspect of one form of the present technology is a method of manufacturing apparatus.

An aspect of certain forms of the present technology is a medical device that is easy to use, e.g. by a person who does not have medical training, by a person who has limited dexterity, vision or by a person with limited experience in using this type of medical device.

Of course, portions of the aspects may form sub-aspects of the present technology. Also, various ones of the sub-aspects and/or aspects may be combined in various manners and also constitute additional aspects or sub-aspects of the present technology.

Other features of the technology will be apparent from consideration of the information contained in the following detailed description, abstract, drawings and claims.

### 3 BRIEF DESCRIPTION OF THE DRAWINGS

The present technology is illustrated by way of example, and not by way of limitation, in the figures of the accompanying drawings, in which like reference numerals refer to similar elements including:

### 3.1 Respiratory Therapy Systems

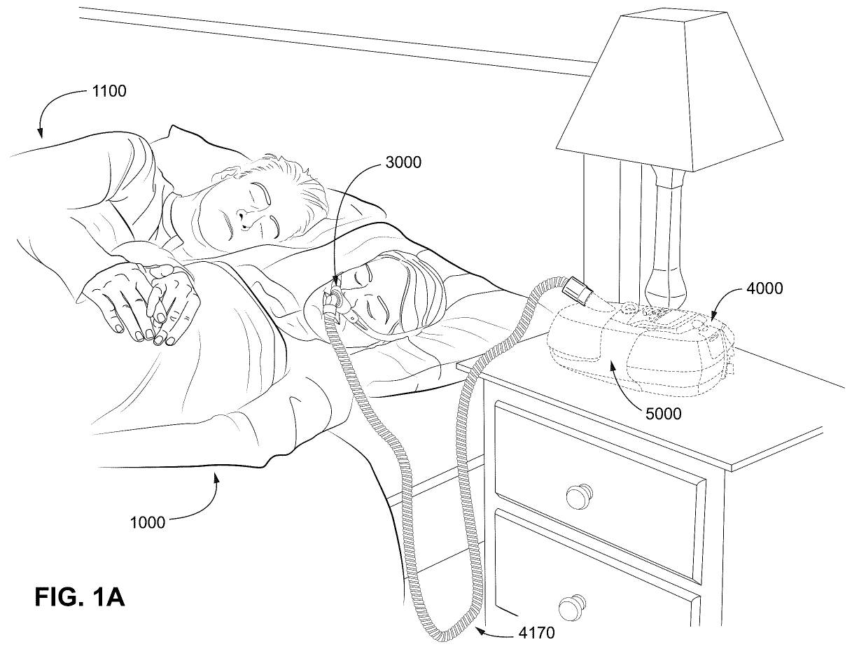

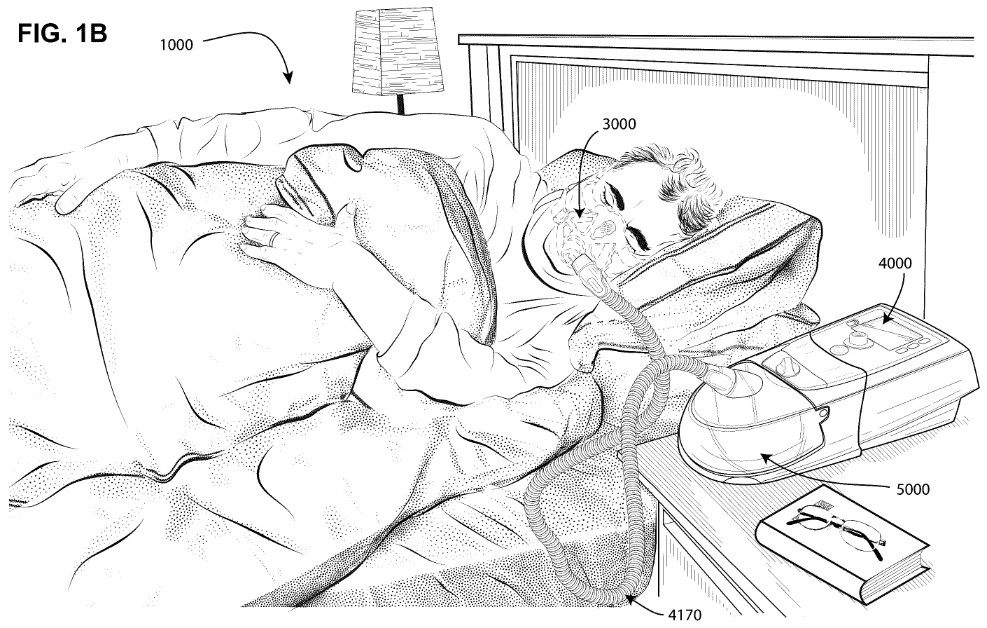

FIG. 1A shows a system including a patient 1000 wearing a patient interface 3000, in the form of nasal pillows, receiving a supply of air at positive pressure from an RPT device 4000. Air from the RPT device 4000 is humidified in a humidifier 5000, and passes along an air circuit 4170 to the patient 1000. A bed partner 1100 is also shown. The patient is sleeping in a supine sleeping position.

FIG. 1B shows a system including a patient 1000 wearing a patient interface 3000, in the form of a full-face mask, receiving a supply of air at positive pressure from an RPT device 4000. Air from the RPT device is humidified in a humidifier 5000, and passes along an air circuit 4170 to the patient 1000. The patient is sleeping in a side sleeping position.

### 3.2 Respiratory System and Facial Anatomy

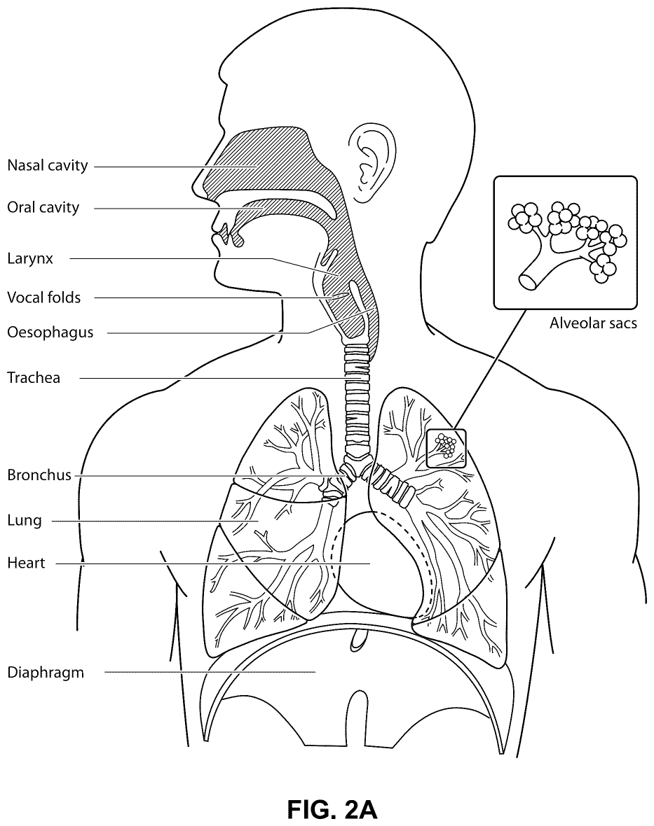

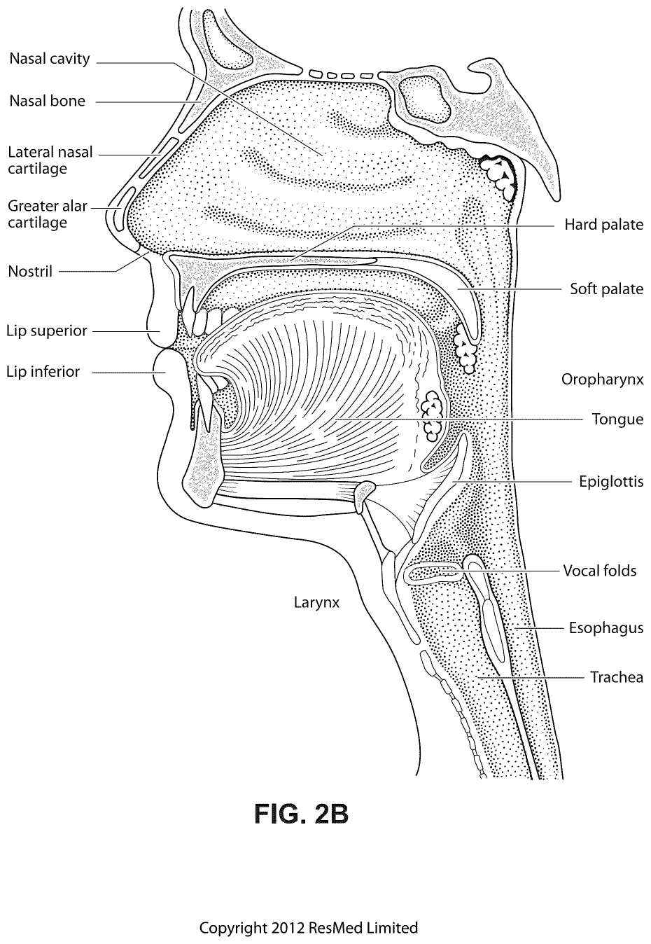

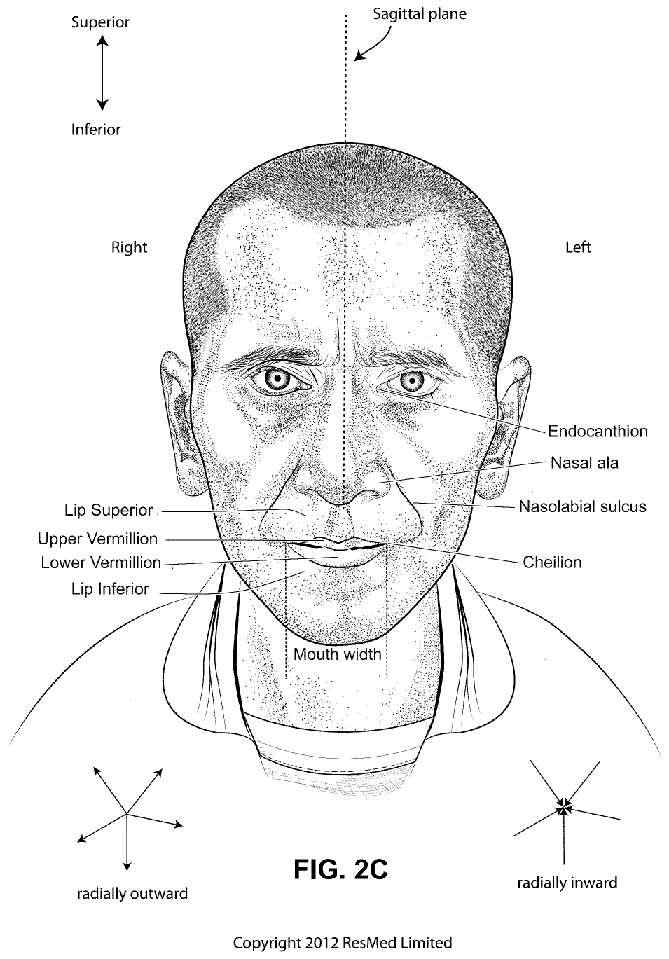

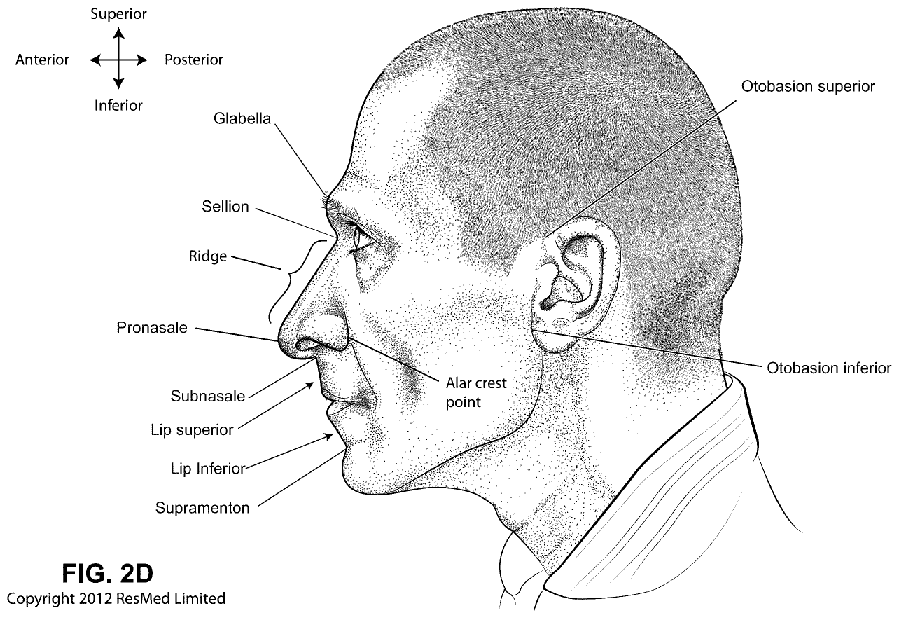

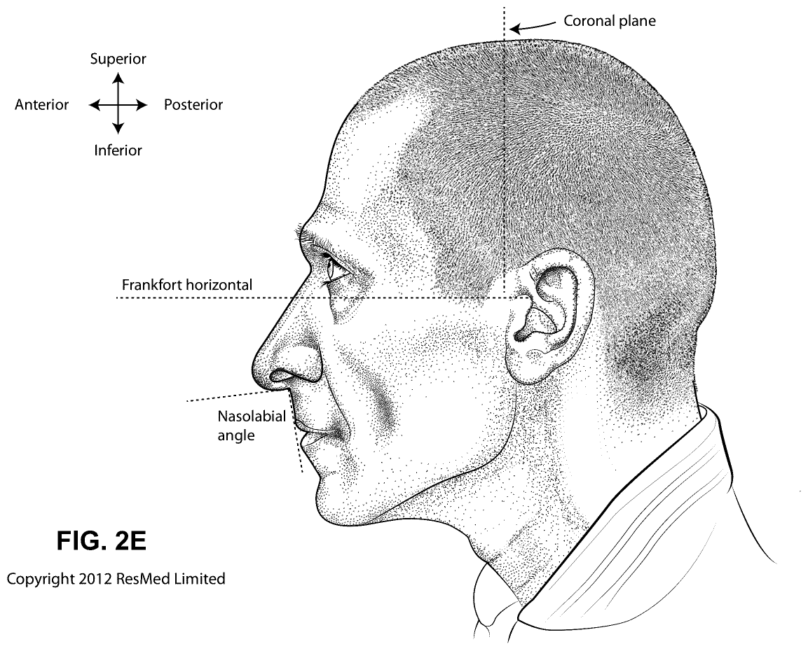

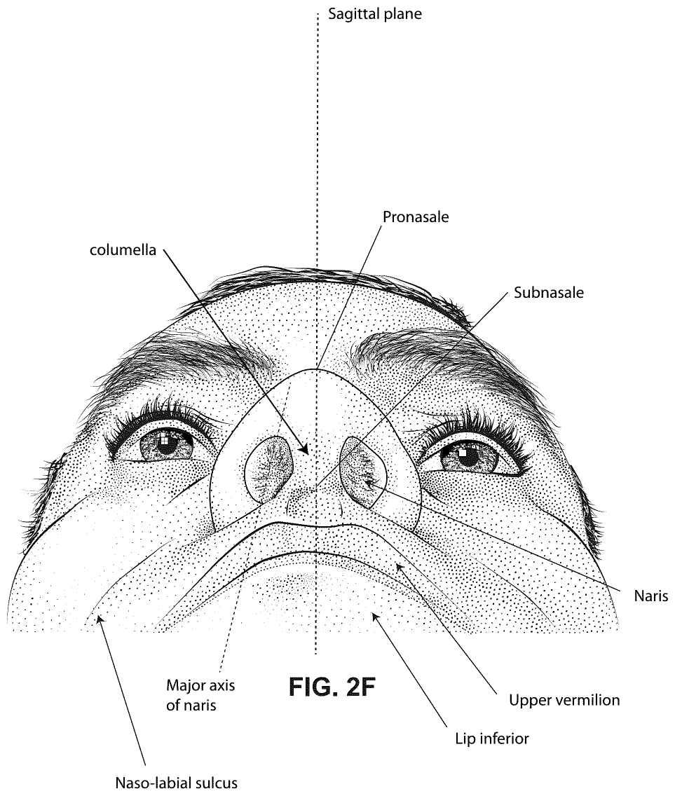

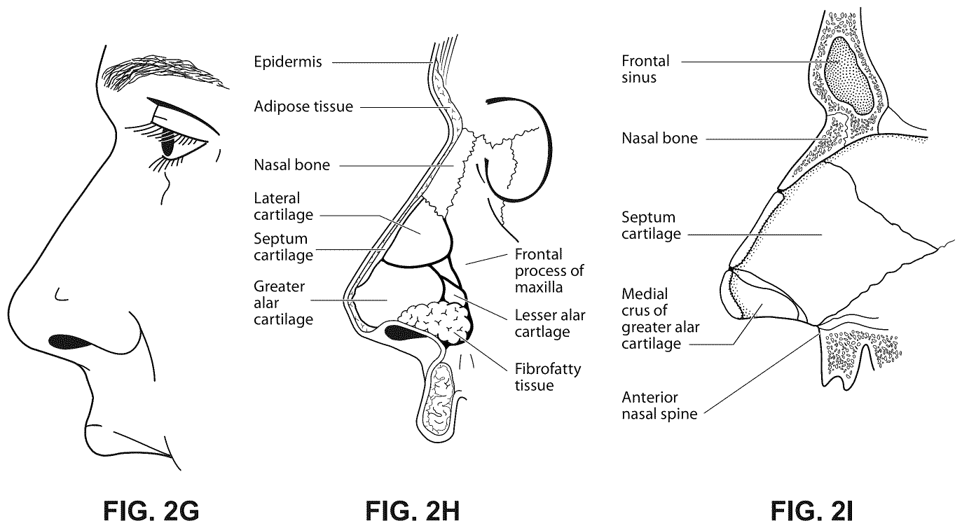

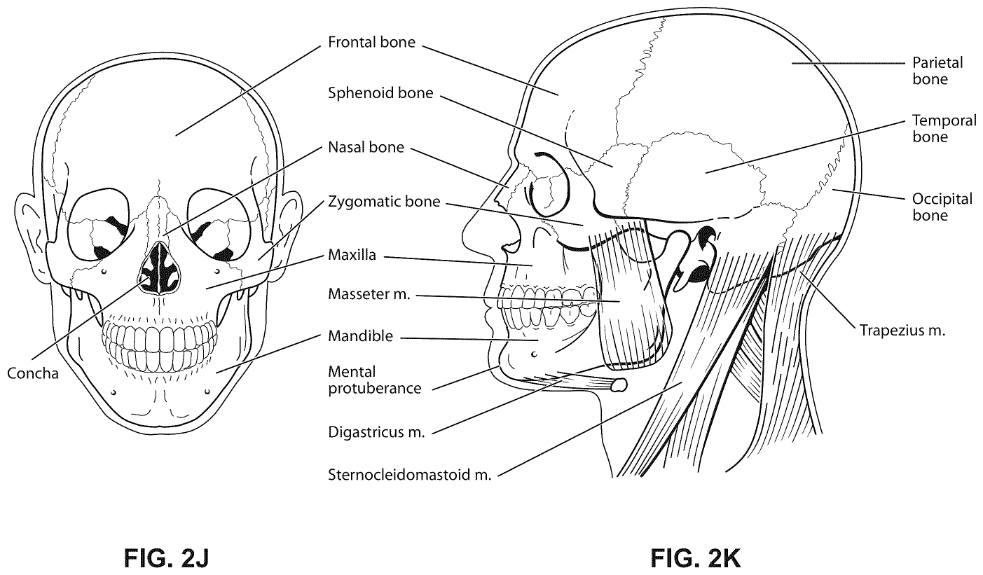

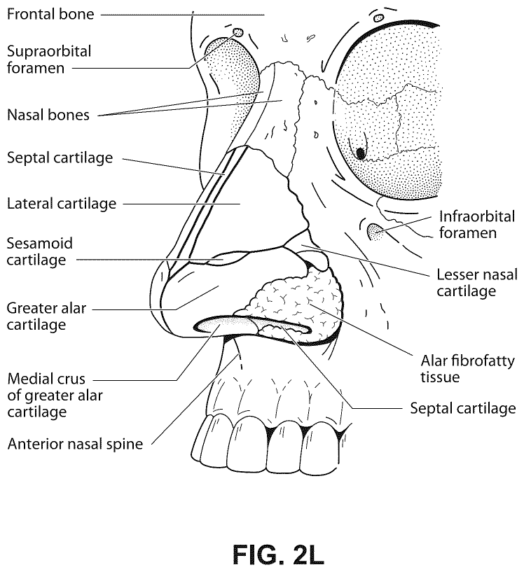

FIG. 2A shows an overview of a human respiratory system including the nasal and oral cavities, the larynx, vocal folds, oesophagus, trachea, bronchus, lung, alveolar sacs, heart and diaphragm.

FIG. 2B shows a view of a human upper airway including the nasal cavity, nasal bone, lateral nasal cartilage, greater alar cartilage, nostril, lip superior, lip inferior, larynx, hard palate, soft palate, oropharynx, tongue, epiglottis, vocal folds, oesophagus and trachea.

FIG. 2C is a front view of a face with several features of surface anatomy identified including the lip superior, upper vermilion, lower vermilion, lip inferior, mouth width, endocanthion, a nasal ala, nasolabial sulcus and cheilion. Also indicated are the directions superior, inferior, radially inward and radially outward.

FIG. 2D is a side view of a head with several features of surface anatomy identified including glabella, sellion, pronasale, subnasale, lip superior, lip inferior, supramenton, nasal ridge, alar crest point, otobasion superior and otobasion inferior. Also indicated are the directions superior & inferior, and anterior & posterior.

FIG. 2E is a further side view of a head. The approximate locations of the Frankfort horizontal and nasolabial angle are indicated. The coronal plane is also indicated.

FIG. 2F shows a base view of a nose with several features identified including naso-labial sulcus, lip inferior, upper Vermilion, naris, subnasale, columella, pronasale, the major axis of a naris and the midsagittal plane.

FIG. 2G shows a side view of the superficial features of a nose.

FIG. 2H shows subcutaneal structures of the nose, including lateral cartilage, septum cartilage, greater alar cartilage, lesser alar cartilage, sesamoid cartilage, nasal bone, epidermis, adipose tissue, frontal process of the maxilla and fibrofatty tissue.

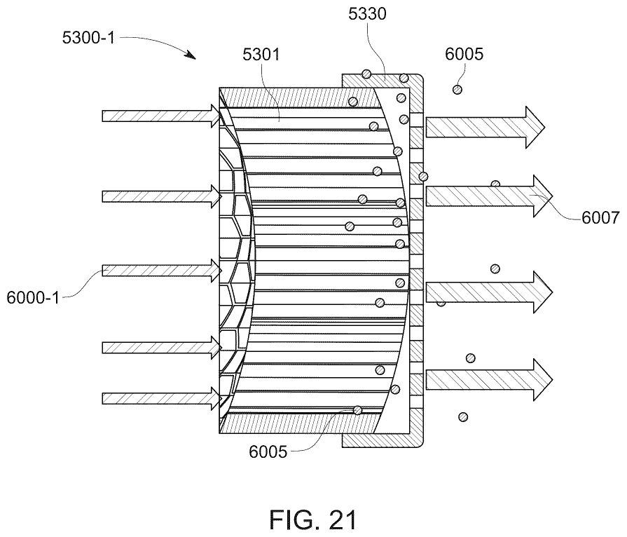

FIG. 21 shows a medial dissection of a nose, approximately several millimeters from the midsagittal plane, amongst other things showing the septum cartilage and medial crus of greater alar cartilage.

FIG. 2J shows a front view of the bones of a skull including the frontal, nasal and zygomatic bones. Nasal concha are indicated, as are the maxilla, and mandible.

FIG. 2K shows a lateral view of a skull with the outline of the surface of a head, as well as several muscles. The following bones are shown: frontal, sphenoid, nasal, zygomatic, maxilla, mandible, parietal, temporal and occipital. The mental protuberance is indicated. The following muscles are shown: digastricus, masseter, sternocleidomastoid and trapezius.

FIG. 2L shows an anterolateral view of a nose.

### 3.3 Patient Interface

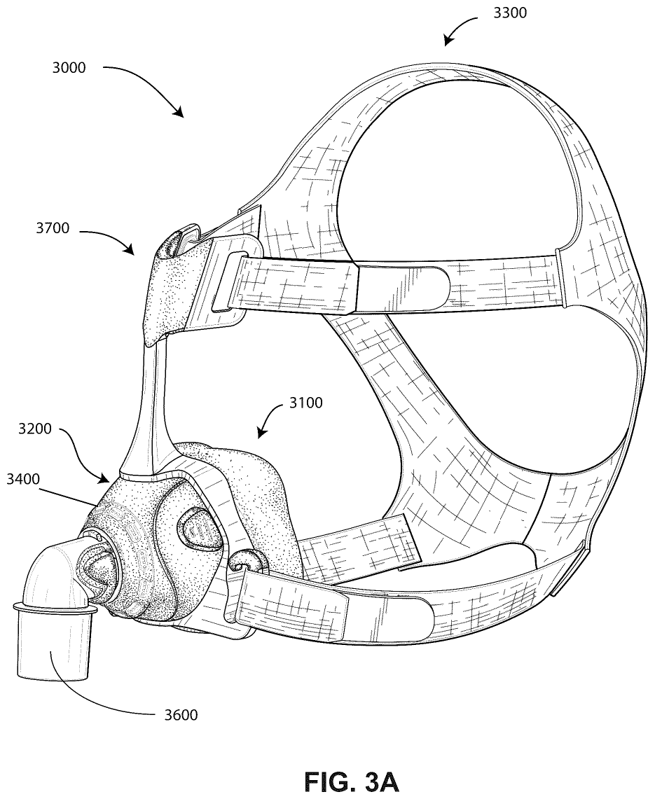

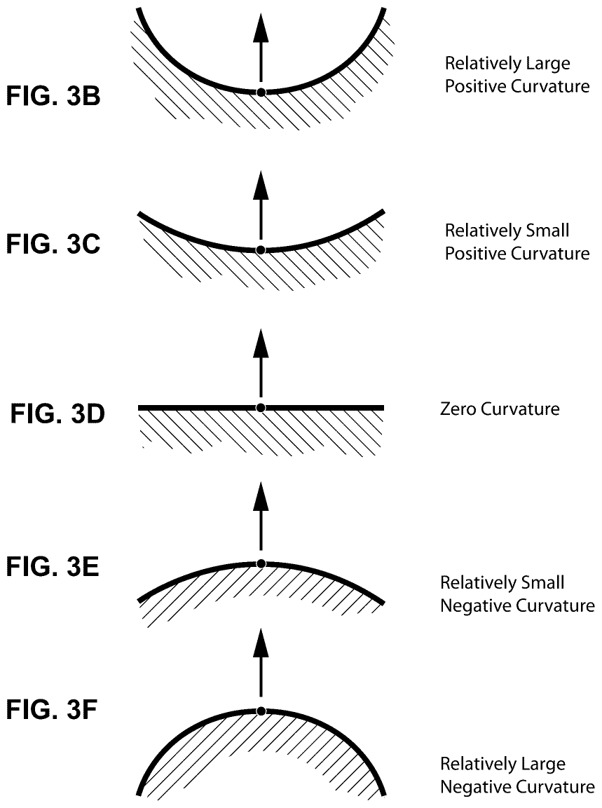

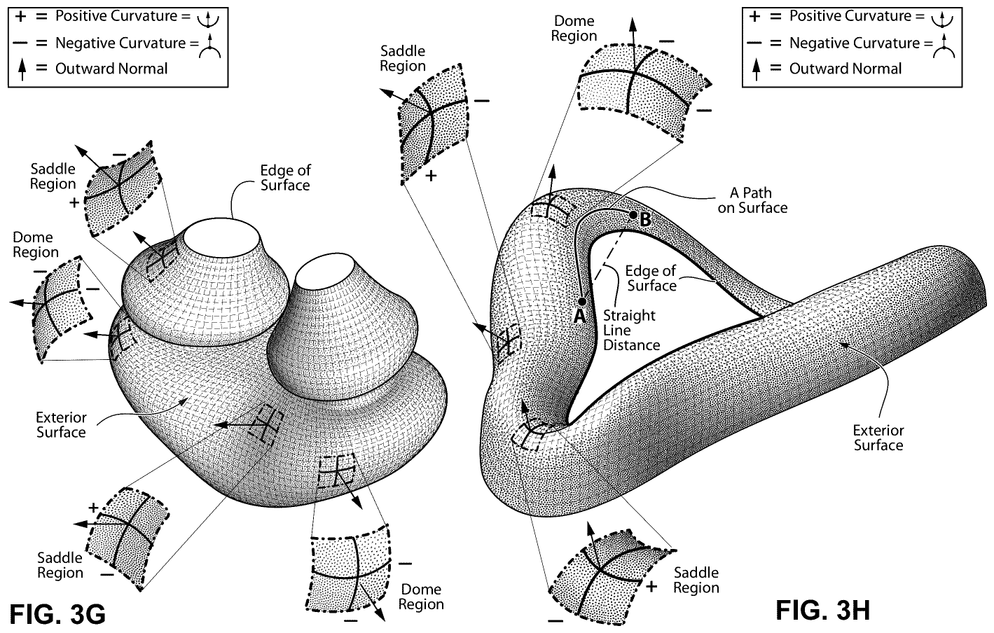

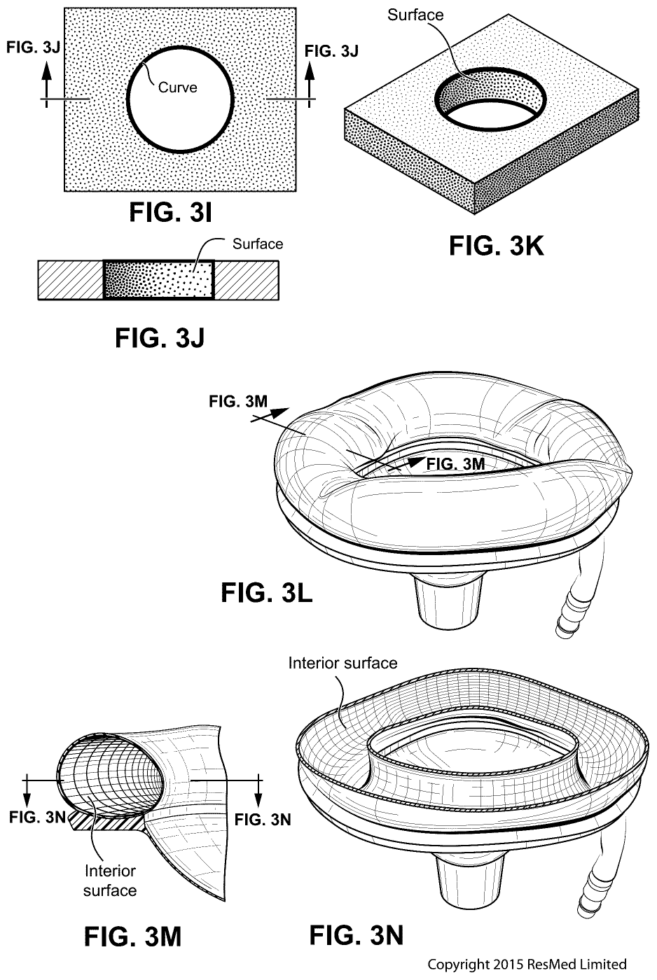

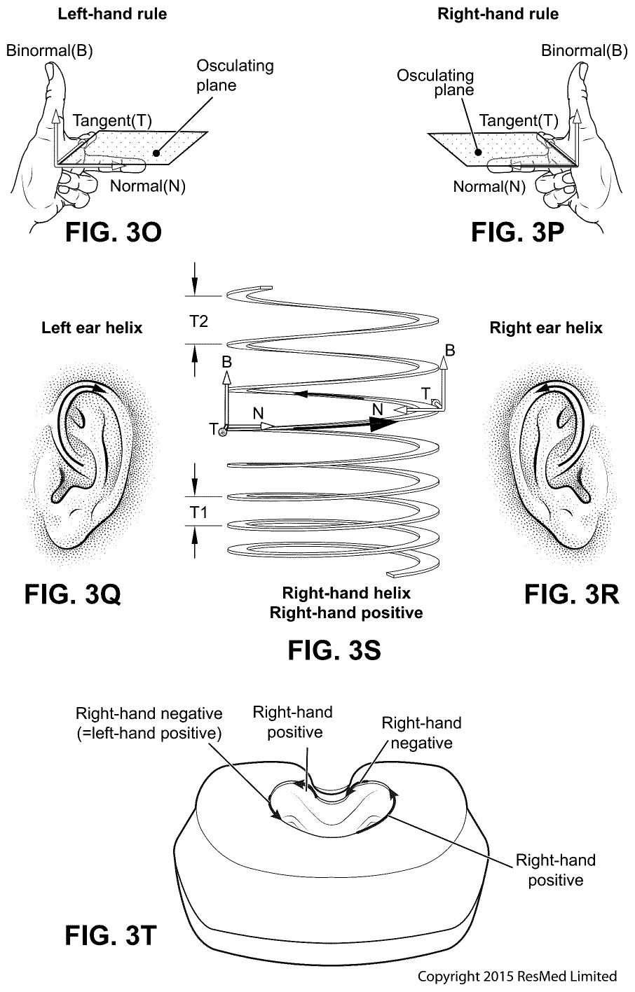

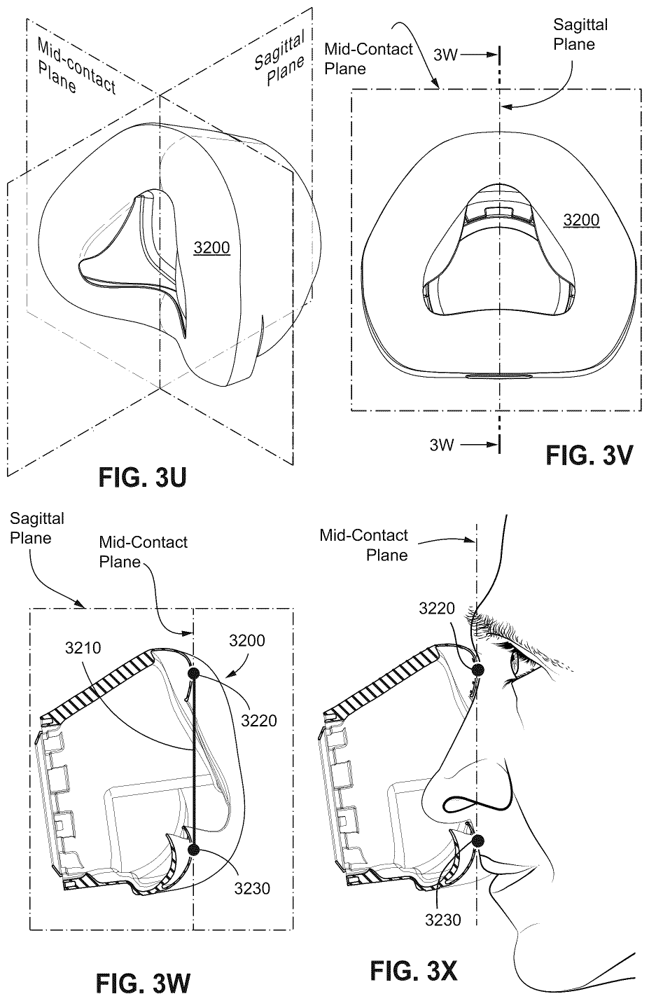

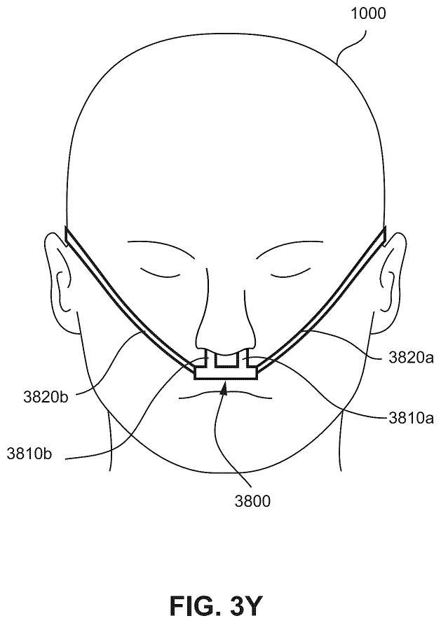

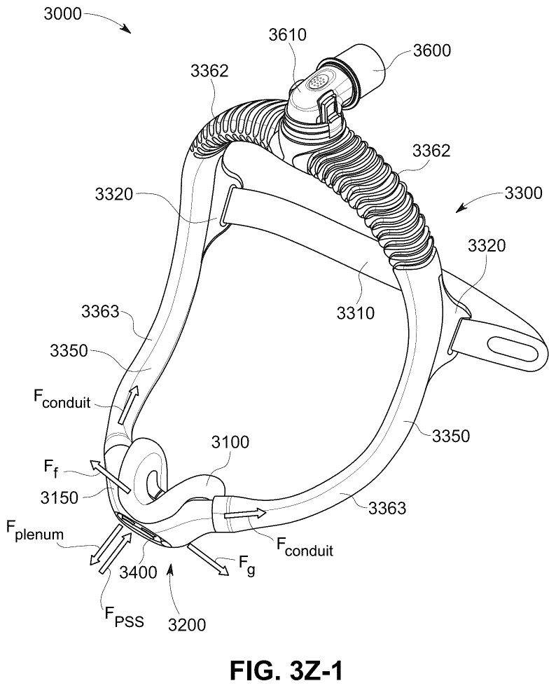

FIG. 3A shows a patient interface in the form of a nasal mask in accordance with one form of the present technology.

FIG. 3B shows a schematic of a cross-section through a structure at a point. An outward normal at the point is indicated. The curvature at the point has a positive sign, and a relatively large magnitude when compared to the magnitude of the curvature shown in FIG. 3C.

FIG. 3C shows a schematic of a cross-section through a structure at a point. An outward normal at the point is indicated. The curvature at the point has a positive sign, and a relatively small magnitude when compared to the magnitude of the curvature shown in FIG. 3B.

FIG. 3D shows a schematic of a cross-section through a structure at a point. An outward normal at the point is indicated. The curvature at the point has a value of zero.

FIG. 3E shows a schematic of a cross-section through a structure at a point. An outward normal at the point is indicated. The curvature at the point has a negative sign, and a relatively small magnitude when compared to the magnitude of the curvature shown in FIG. 3F.

FIG. 3F shows a schematic of a cross-section through a structure at a point. An outward normal at the point is indicated. The curvature at the point has a negative sign, and a relatively large magnitude when compared to the magnitude of the curvature shown in FIG. 3E.

FIG. 3G shows a cushion for a mask that includes two pillows. An exterior surface of the cushion is indicated. An edge of the surface is indicated. Dome and saddle regions are indicated.

FIG. 3H shows a cushion for a mask. An exterior surface of the cushion is indicated. An edge of the surface is indicated. A path on the surface between points A and B is indicated. A straight line distance between A and B is indicated. Two saddle regions and a dome region are indicated.

FIG. 3I shows the surface of a structure, with a one dimensional hole in the surface. The illustrated plane curve forms the boundary of a one dimensional hole.

FIG. 3J shows a cross-section through the structure of FIG. 3I. The illustrated surface bounds a two-dimensional hole in the structure of FIG. 3I.

FIG. 3K shows a perspective view of the structure of FIG. 3I, including the two-dimensional hole and the one dimensional hole. Also shown is the surface that bounds a two-dimensional hole in the structure of FIG. 3I.

FIG. 3L shows a mask having an inflatable bladder as a cushion.

FIG. 3M shows a cross-section through the mask of FIG. 3L, and shows the interior surface of the bladder. The interior surface bounds the two dimensional hole in the mask.

FIG. 3N shows a further cross-section through the mask of FIG. 3L. The interior surface is also indicated.

FIG. 3O illustrates a left-hand rule.

FIG. 3P illustrates a right-hand rule.

FIG. 3Q shows a left ear, including the left ear helix.

FIG. 3R shows a right ear, including the right ear helix.

FIG. 3S shows a right-hand helix.

FIG. 3T shows a view of a mask, including the sign of the torsion of the space curve defined by the edge of the sealing membrane in different regions of the mask.

FIG. 3U shows a view of a plenum chamber 3200 showing a sagittal plane and a mid-contact plane.

FIG. 3V shows a view of a posterior of the plenum chamber of FIG. 3U. The direction of the view is normal to the mid-contact plane. The sagittal plane in FIG. 3V bisects the plenum chamber into left-hand and right-hand sides.

FIG. 3W shows a cross-section through the plenum chamber of FIG. 3V, the cross-section being taken at the sagittal plane shown in FIG. 3V. A ‘mid-contact’ plane is shown. The mid-contact plane is perpendicular to the sagittal plane. The orientation of the mid-contact plane corresponds to the orientation of a chord 3210 which lies on the sagittal plane and just touches the cushion of the plenum chamber at two points on the sagittal plane: a superior point 3220 and an inferior point 3230. Depending on the geometry of the cushion in this region, the mid-contact plane may be a tangent at both the superior and inferior points.

FIG. 3X shows the plenum chamber 3200 of FIG. 3U in position for use on a face. The sagittal plane of the plenum chamber 3200 generally coincides with the midsagittal plane of the face when the plenum chamber is in position for use. The mid-contact plane corresponds generally to the ‘plane of the face’ when the plenum chamber is in position for use. In FIG. 3X the plenum chamber 3200 is that of a nasal mask, and the superior point 3220 sits approximately on the sellion, while the inferior point 3230 sits on the lip superior.

FIG. 3Y shows a patient interface in the form of a nasal cannula in accordance with one form of the present technology.

FIG. 3Z-1 shows a patient interface having conduit headgear and forces acting on the patient interface while in use, in accordance with one form of the present technology.

### 3.4 RPT Device

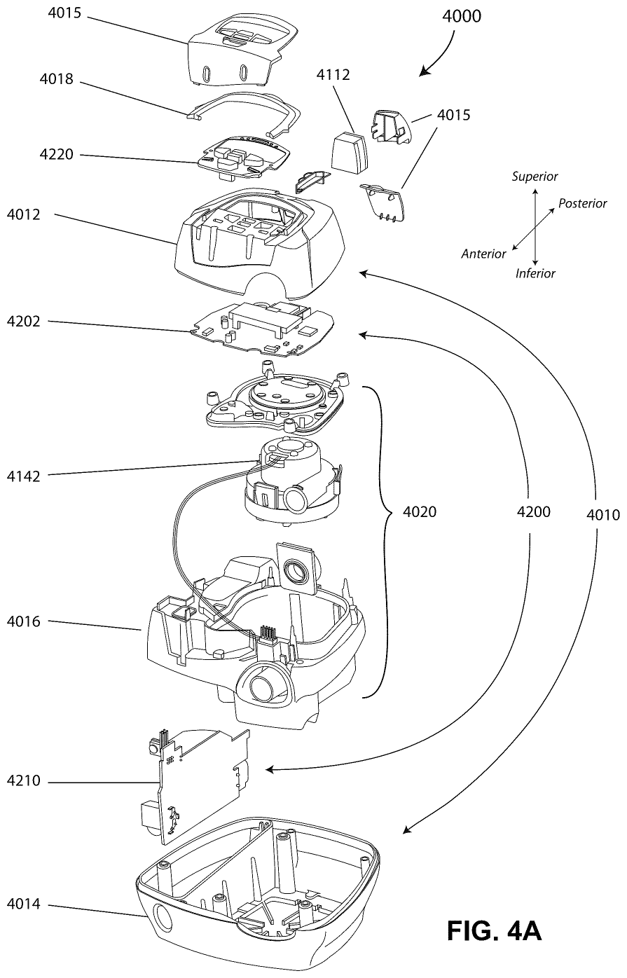

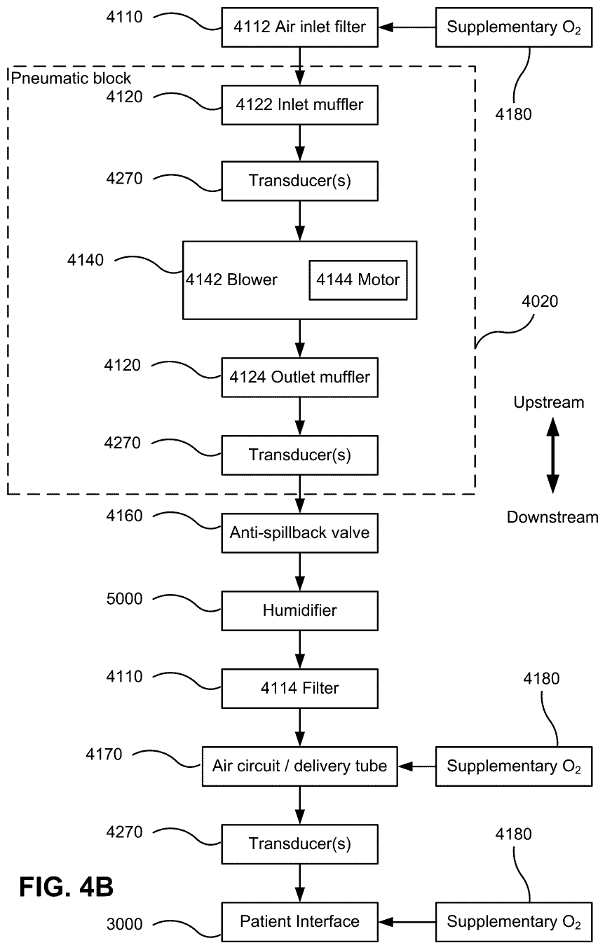

FIG. 4A shows an RPT device in accordance with one form of the present technology.

FIG. 4B is a schematic diagram of the pneumatic path of an RPT device in accordance with one form of the present technology. The directions of upstream and downstream are indicated with reference to the blower and the patient interface. The blower is defined to be upstream of the patient interface and the patient interface is defined to be downstream of the blower, regardless of the actual flow direction at any particular moment. Items which are located within the pneumatic path between the blower and the patient interface are downstream of the blower and upstream of the patient interface.

### 3.5 Humidifier

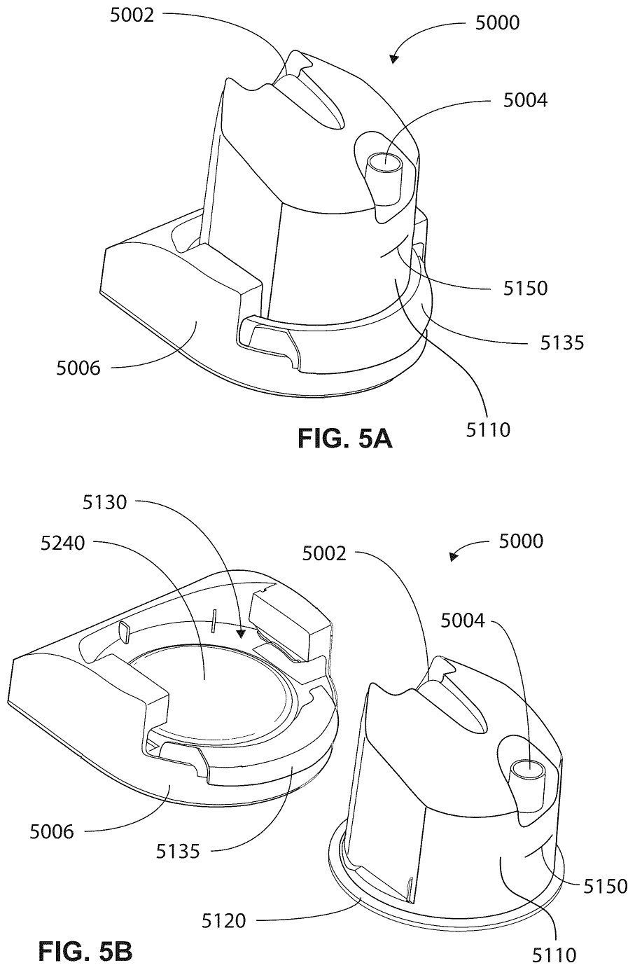

FIG. 5A shows an isometric view of a humidifier in accordance with one form of the present technology.

FIG. 5B shows an isometric view of a humidifier in accordance with one form of the present technology, showing a humidifier reservoir 5110 removed from the humidifier reservoir dock 5130.

### 3.6 Breathing Waveforms

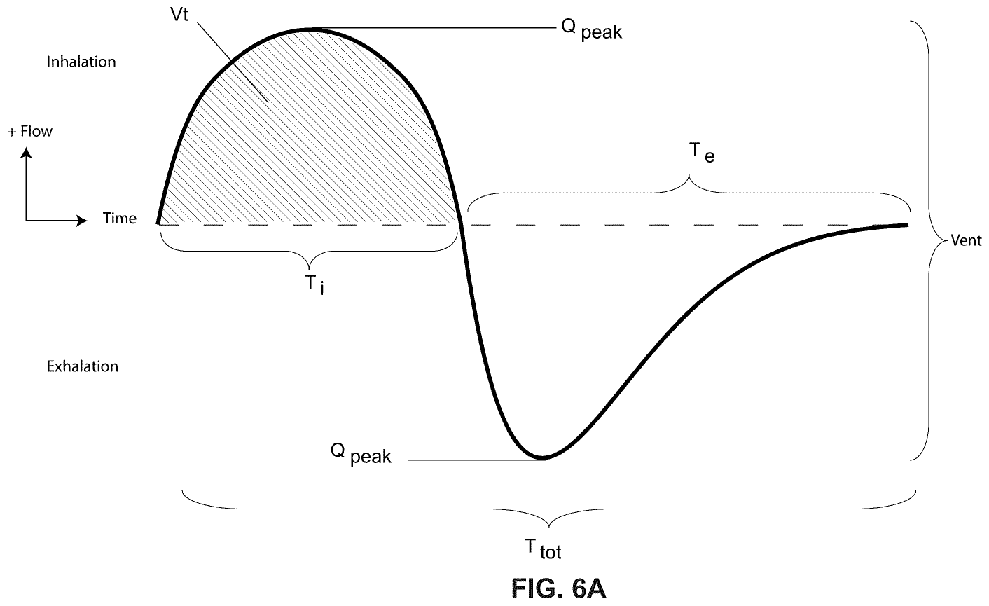

FIG. 6A shows a model typical breath waveform of a person while sleeping.

### 3.7 Modularity

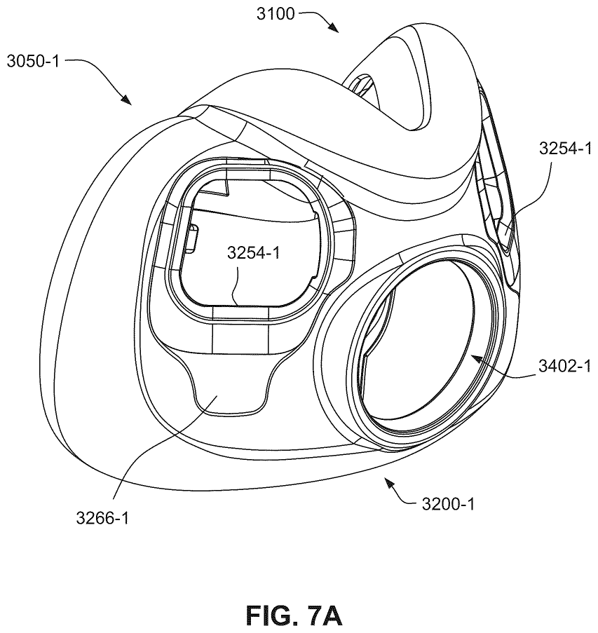

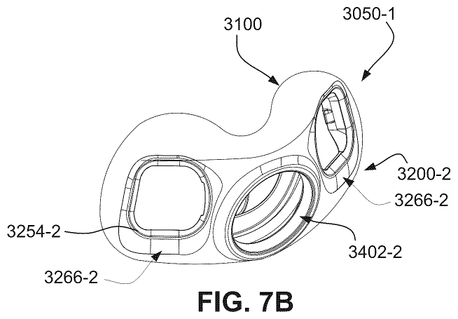

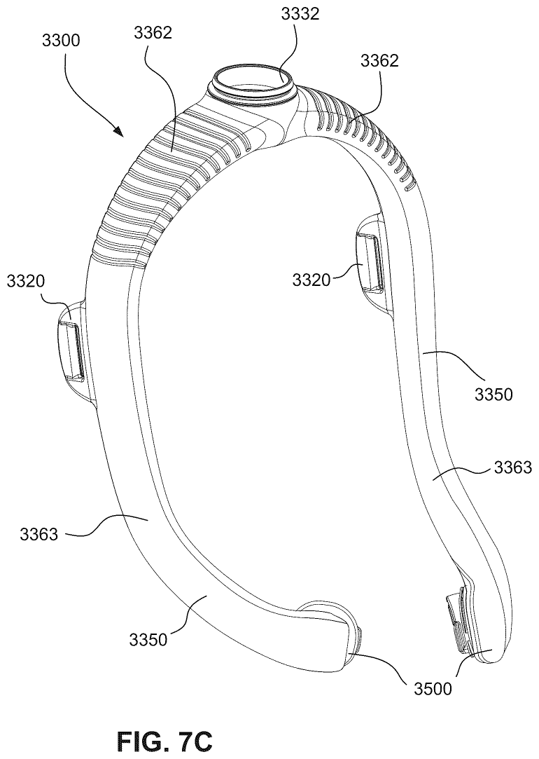

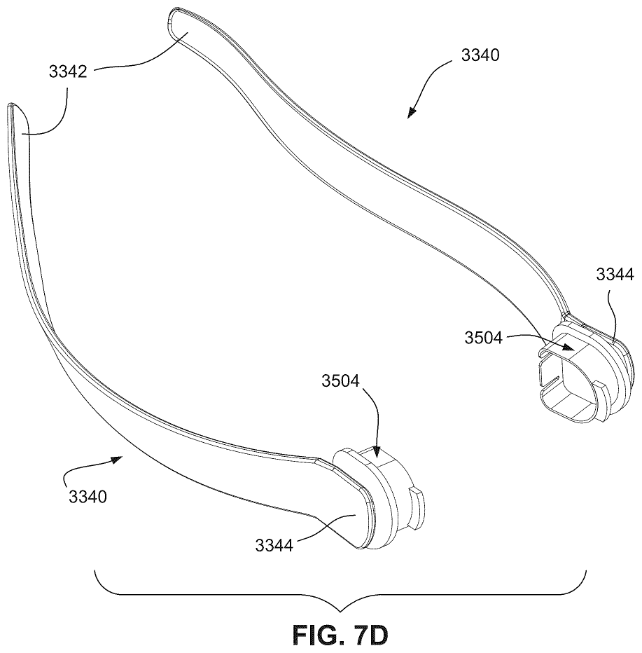

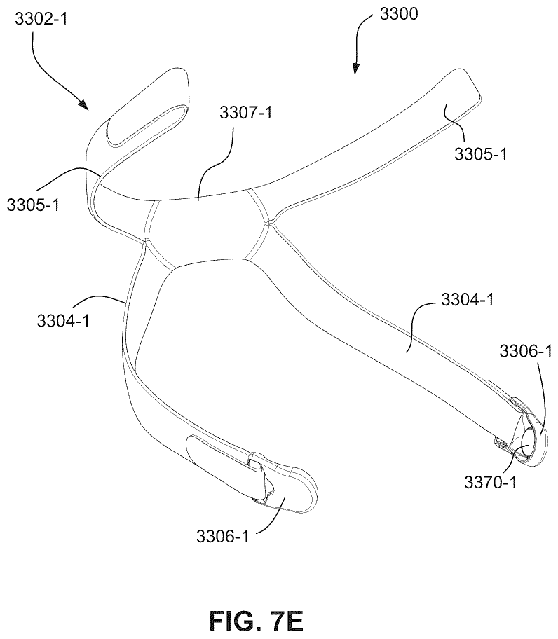

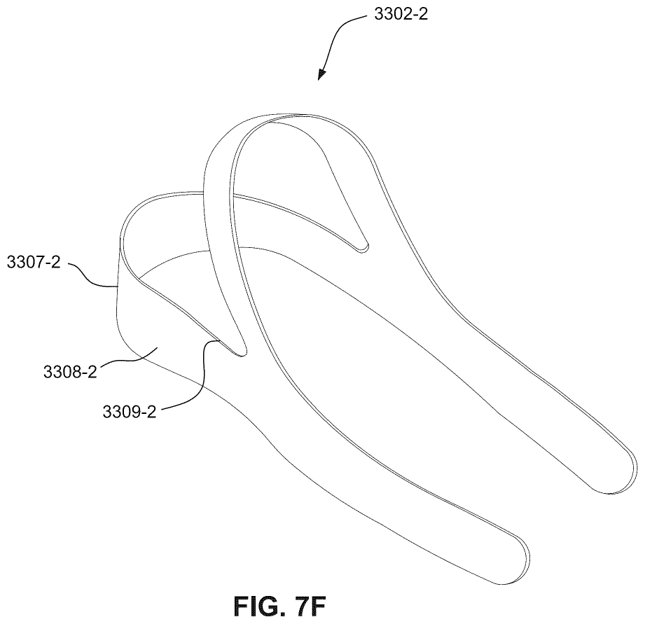

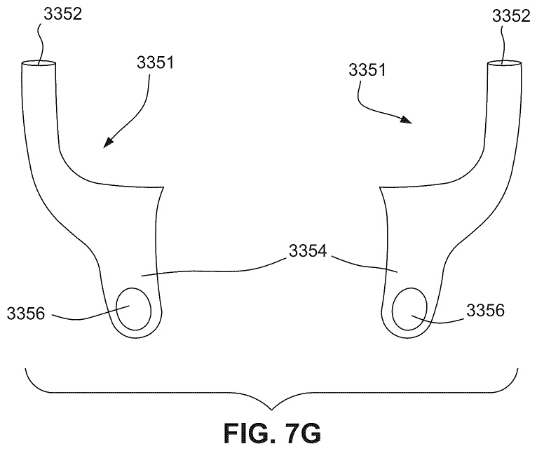

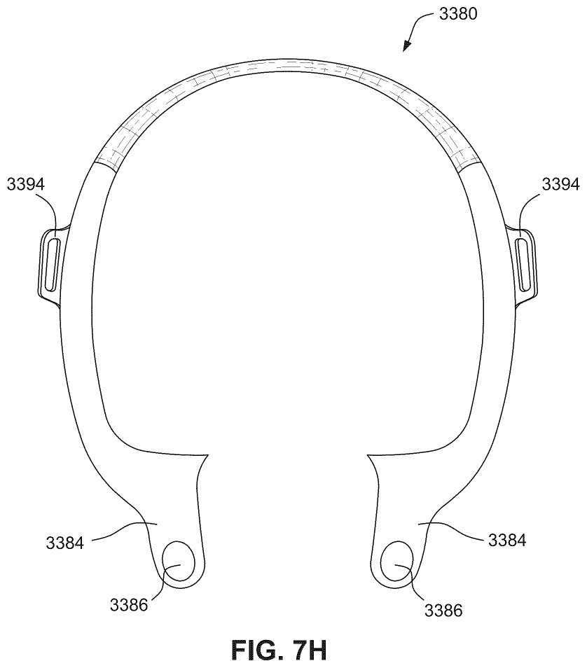

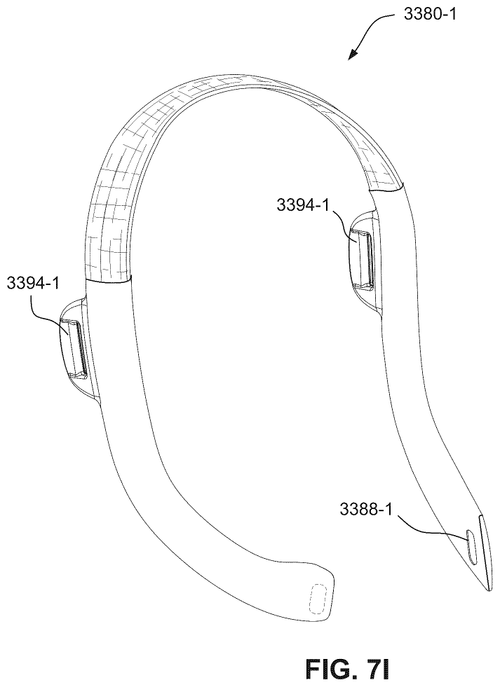

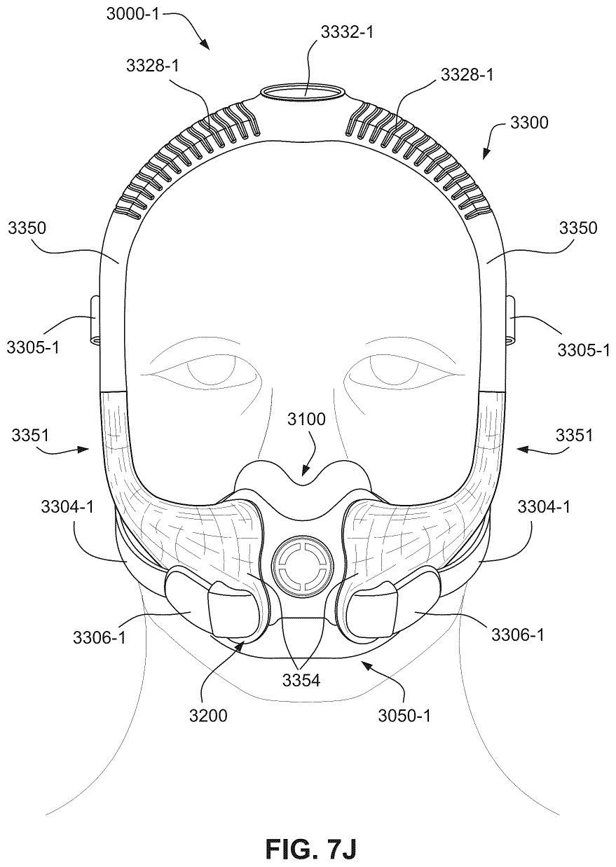

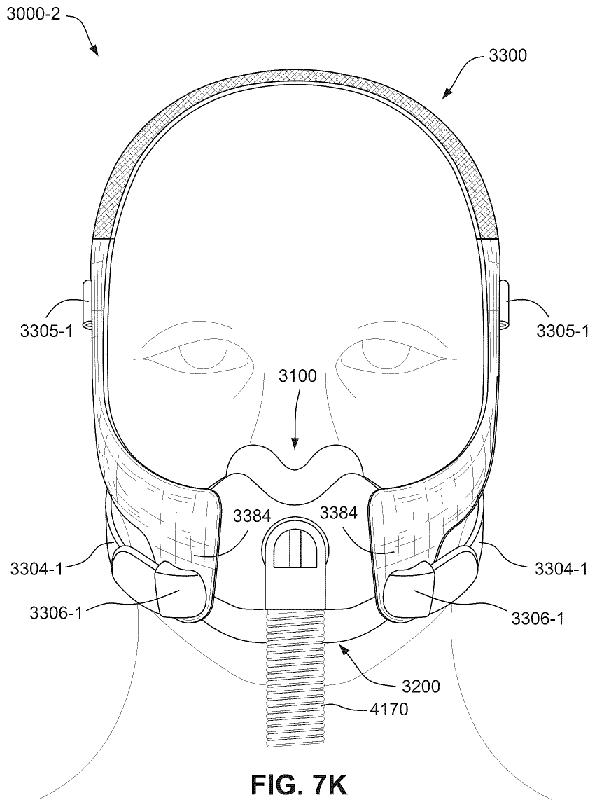

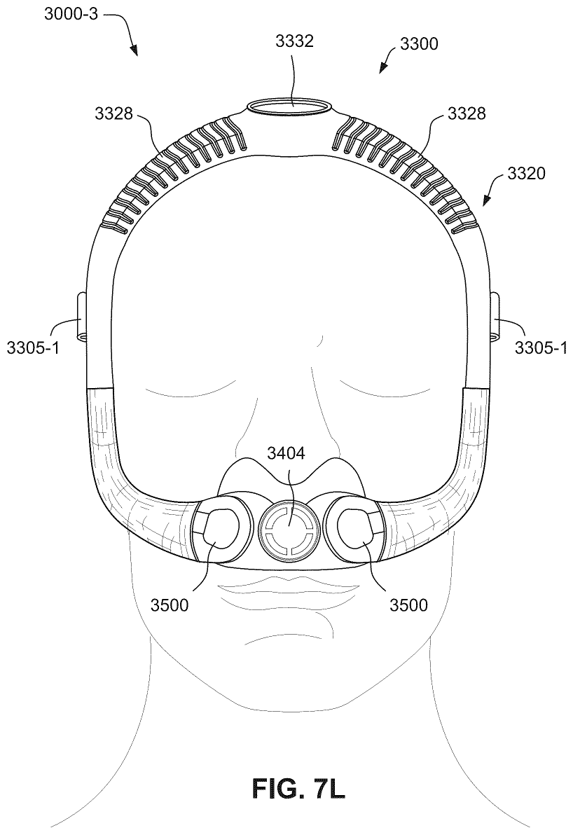

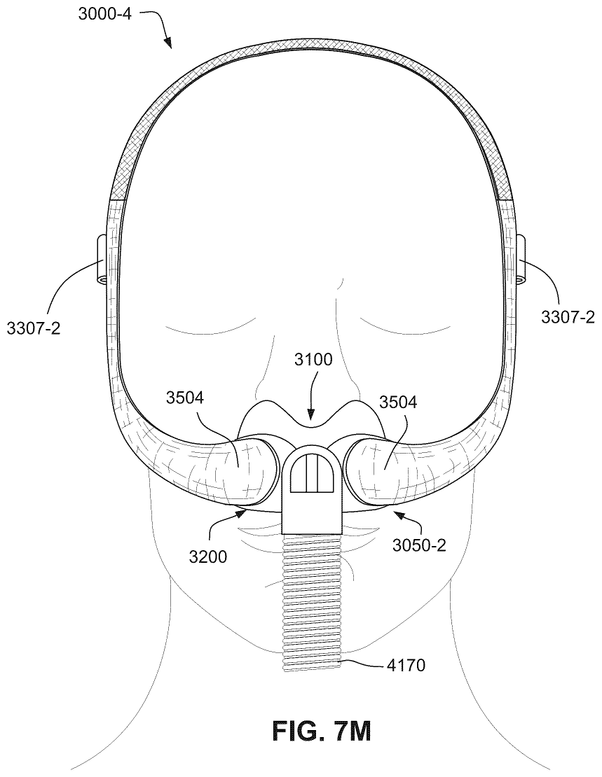

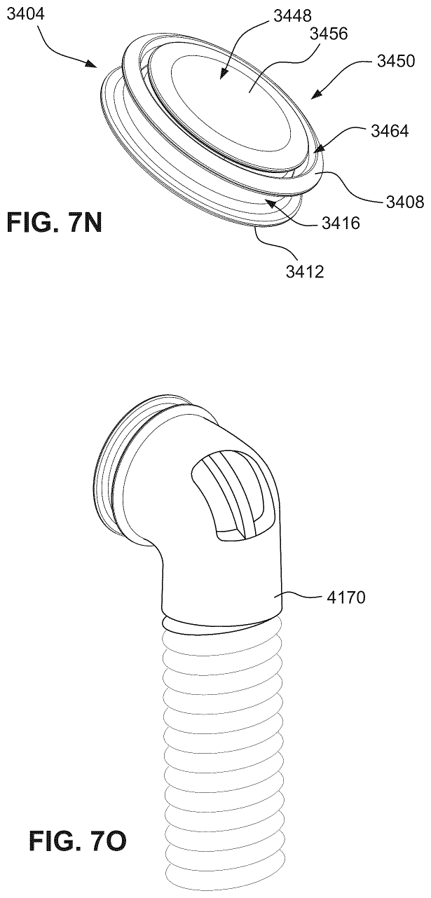

FIG. 7A shows a perspective view of a cushion of a patient interface configured to be worn by a patient and convey pressurized air to the patient's nose and the patient's mouth.

FIG. 7B shows a perspective view of a cushion of a patient interface configured to be worn by a patient and convey pressurized air to the patient's nose.

FIG. 7C shows a perspective view of tubes usable with either the cushion of FIG. 7A or the cushion of FIG. 7B.

FIG. 7D shows a perspective view of rigidiser arms usable with either the cushion of FIG. 7A of the cushion of FIG. 7B.

FIG. 7E shows a perspective view of headgear straps usable with the cushion of FIG. 7A.

FIG. 7F shows a perspective view of headgear straps usable with the cushion of FIG. 7B.

FIG. 7G shows a front view of a pair of sleeves that is removably fitted to either the tubes of FIG. 7C or the rigidiser arms of FIG. 7D.

FIG. 7H shows a front view of a full sleeve that is removably fitted to the rigidiser arms of FIG. 7D.

FIG. 7I shows a front perspective view of yet another alternate form of a full sleeve that is removably fitted to the rigidiser arms of FIG. 7D.

FIG. 7J is a front view of a patient wearing the cushion of FIG. 7A connected to the tubes of FIG. 7C, the headgear straps of FIG. 7E, and the sleeves of FIG. 7G.

FIG. 7K is a front view of a patient wearing the cushion of FIG. 7A connected to the rigidiser arms of FIG. 7D, the headgear straps of FIG. 7E, and the sleeve of FIG. 7H.

FIG. 7L is a front view of a patient wearing the cushion of FIG. 7B connected to the conduit headgear of FIG. 7C, and the headgear straps of FIG. 7F.

FIG. 7M is a front view of a patient wearing the cushion of FIG. 7B connected to the rigidisier arms of FIG. 7D, the headgear straps of FIG. 7F, and the sleeve of FIG. 7I.

FIG. 7N is an isolated perspective view of the vent of FIG. 7L.

FIG. 7O is an isolated perspective view of a portion of the air circuit of FIG. 7M.

### 3.8 Patient Interfaces & HMES

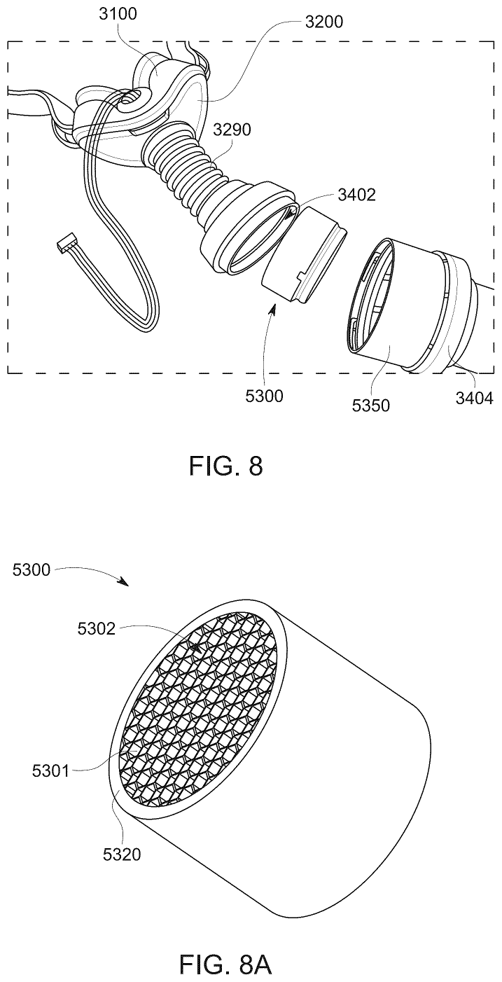

FIG. 8. shows a nasal mask including an HME within the patient interface.

FIG. 8A shows an example of heat and moisture exchange component for use in a patient interface.

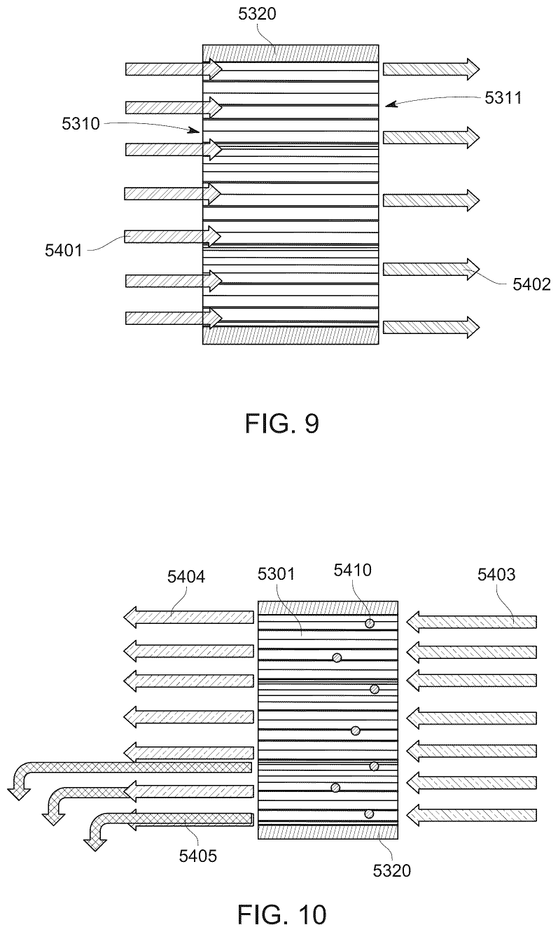

FIG. 9 shows the process of inhalation through heat and moisture exchange component in a patient interface.

FIG. 10 shows the process of exhalation through heat and moisture exchange component in a patient interface.

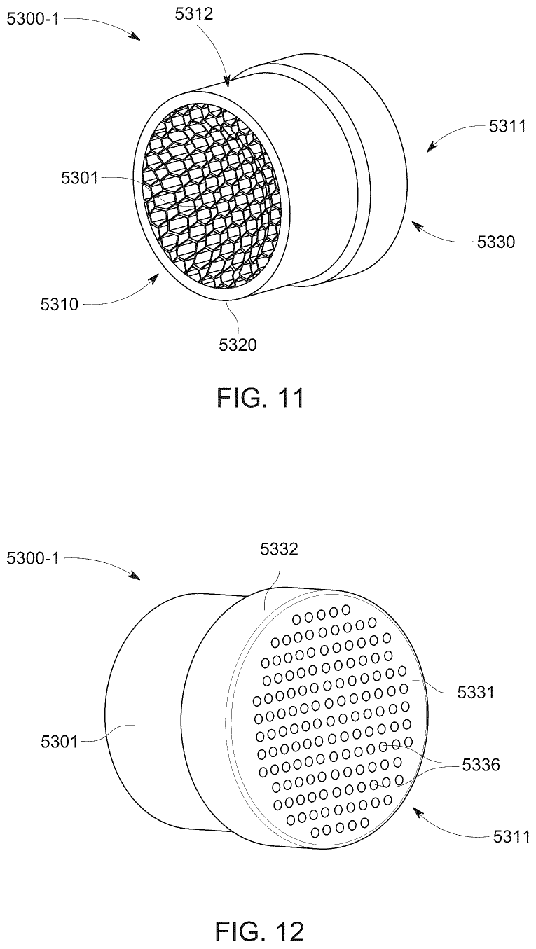

FIG. 11 is a perspective view of a non-patient facing side of an HME with a heat and moisture exchange component and a thermally conductive member.

FIG. 12 is a perspective view of a patient facing side of an HME with a heat and moisture exchange component and a thermally conductive member.

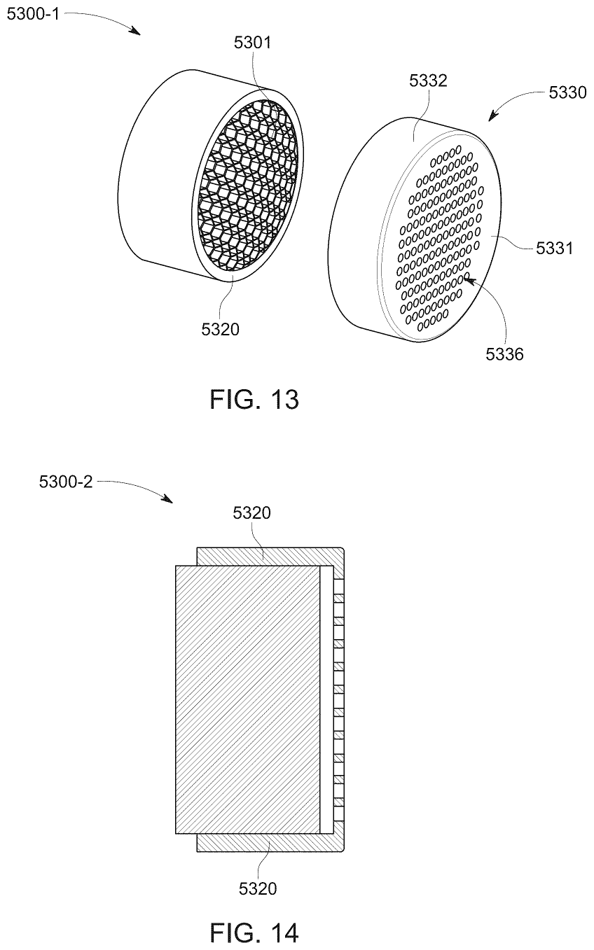

FIG. 13 is an exploded view of the HME with a heat and moisture exchange component and a thermally conductive member.

FIG. 14 shows an example of an HME including a thermally conductive member in the form of a casing structure.

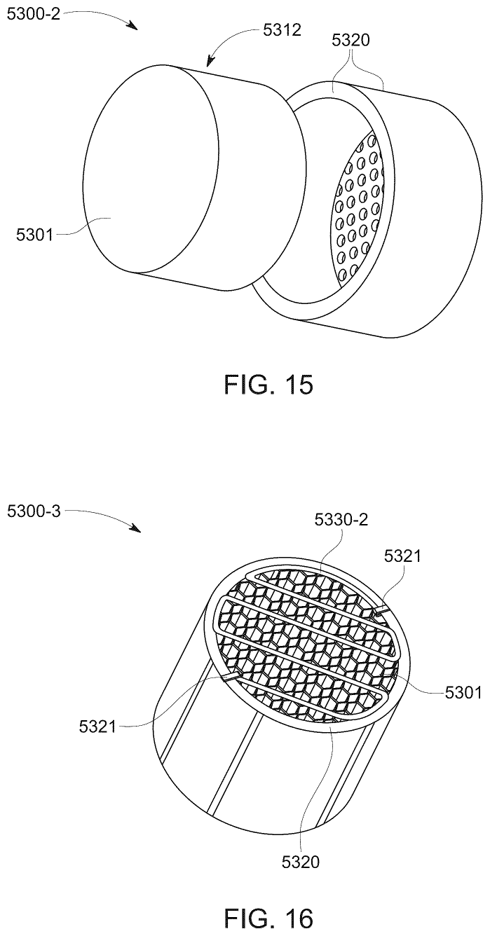

FIG. 15 shows an exploded view of the including a thermally conductive member in the form of a casing structure.

FIG. 16 shows a perspective view of a patient facing side of an HME with a heat and moisture exchange component and a thermally conductive member in an alternative example of the invention.

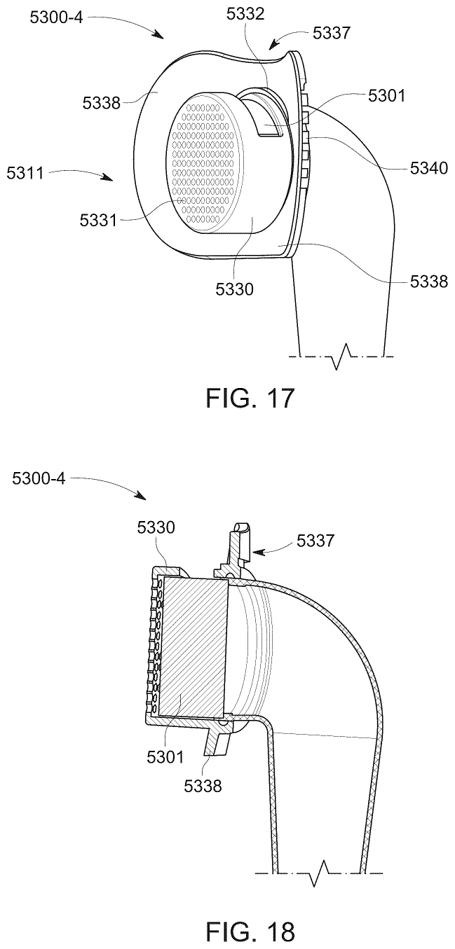

FIG. 17 shows a perspective view of a patient facing side of an HME with a heat and moisture exchange component, thermally conductive member and projection structure in an alternative example of the invention.

FIG. 18 shows a cross section view of an HME with a heat and moisture exchange component, thermally conductive member and integrally formed projection structure in an alternative example of the invention.

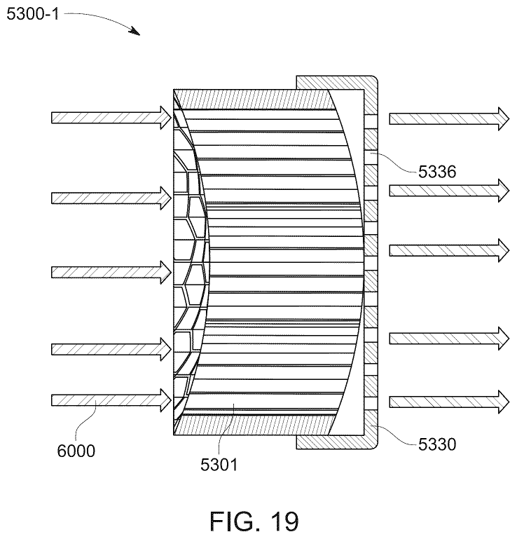

FIG. 19 shows the start of inhalation of air through an HME comprising an heat and moisture exchange component and a thermally conductive element.

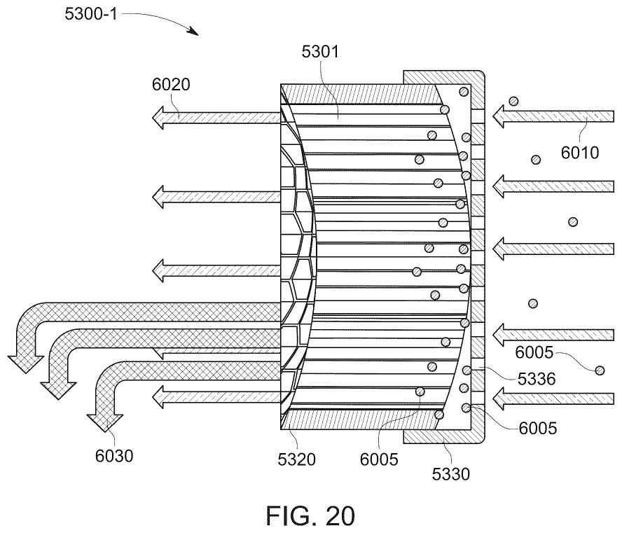

FIG. 20 shows exhalation of air through and HME comprising a heat and moisture exchange component and a thermally conductive element.

FIG. 21 shows secondary inhalation of air through and HME comprising an heat and moisture exchange component and a thermally conductive element.

### 4 DETAILED DESCRIPTION OF EXAMPLES OF THE TECHNOLOGY

Before the present technology is described in further detail, it is to be understood that the technology is not limited to the particular examples described herein, which may vary. It is also to be understood that the terminology used in this disclosure is for the purpose of describing only the particular examples discussed herein, and is not intended to be limiting.

The following description is provided in relation to various examples which may share one or more common characteristics and/or features. It is to be understood that one or more features of any one example may be combinable with one or more features of another example or other examples. In addition, any single feature or combination of features in any of the examples may constitute a further example.

### 4.1 Therapy

In one form, the present technology comprises a method for treating a respiratory disorder comprising applying positive pressure to the entrance of the airways of a patient 1000.

In certain examples of the present technology, a supply of air at positive pressure is provided to the nasal passages of the patient via one or both nares.

In certain examples of the present technology, mouth breathing is limited, restricted or prevented.

### 4.2 Respiratory Therapy Systems

In one form, the present technology comprises a respiratory therapy system for treating a respiratory disorder. The respiratory therapy system may comprise an RPT device 4000 for supplying a flow of air to the patient 1000 via an air circuit 4170 and a patient interface 3000 or 3800.

### 4.3 Patient Interface

A non-invasive patient interface 3000, such as that shown in FIG. 3A, in accordance with one aspect of the present technology comprises the following functional aspects: a seal-forming structure 3100, a plenum chamber 3200, a positioning and stabilising structure 3300, a vent 3400, one form of connection port 3600 for connection to air circuit 4170, and a forehead support 3700. In some forms a functional aspect may be provided by one or more physical components. In some forms, one physical component may provide one or more functional aspects. In use the seal-forming structure 3100 is arranged to surround an entrance to the airways of the patient so as to maintain positive pressure at the entrance(s) to the airways of the patient 1000. The sealed patient interface 3000 is therefore suitable for delivery of positive pressure therapy.

As shown in FIG. 3Z-1, a non-invasive patient interface 3000 in accordance with another aspect of the present technology comprises the following functional aspects: a seal-forming structure 3100, a plenum chamber 3200, a positioning and stabilising structure 3300, a vent 3400 and one form of connection port 3600 for connection to an air circuit (such as the air circuit 4170 shown in FIGS. 1A-1B). The plenum chamber 3200 may be formed of one or more modular components (e.g., a cushion module 3150 together with the seal-forming structure 3100) in the sense that it or they can be replaced with different components, for example components of a different size.

An unsealed patient interface 3800, in the form of a nasal cannula, includes nasal prongs 3810a, 3810b which can deliver air to respective nares of the patient 1000 via respective orifices in their tips. Such nasal prongs do not generally form a seal with the inner or outer skin surface of the nares. This type of interface results in one or more gaps that are present in use by design (intentional) but they are typically not fixed in size such that they may vary unpredictably by movement during use. This can present a complex pneumatic variable for a respiratory therapy system when pneumatic control and/or assessment is implemented, unlike other types of mask-based respiratory therapy systems. The air to the nasal prongs may be delivered by one or more air supply lumens 3820a, 3820b that are coupled with the nasal cannula-type unsealed patient interface 3800. The lumens 3820a, 3820b lead from the nasal cannula-type unsealed patient interface 3800 to a respiratory therapy device via an air circuit. The unsealed patient interface 3800 is particularly suitable for delivery of flow therapies, in which the RPT device generates the flow of air at controlled flow rates rather than controlled pressures. The “vent” or gap at the unsealed patient interface 3800, through which excess airflow escapes to ambient, is the passage between the end of the prongs 3810a and 3810b of the nasal cannula-type unsealed patient interface 3800 via the patient's nares to atmosphere.

If a patient interface is unable to comfortably deliver a minimum level of positive pressure to the airways, the patient interface may be unsuitable for respiratory pressure therapy.

The patient interface 3000 in accordance with one form of the present technology is constructed and arranged to be able to provide a supply of air at a positive pressure above the ambient, for example at least 2, 4, 6, 10, or 20 cmH2O with respect to ambient.

### 4.3.1 Seal-Forming Structure

In one form of the present technology, a seal-forming structure 3100 provides a target seal-forming region, and may additionally provide a cushioning function. The target seal-forming region is a region on the seal-forming structure 3100 where sealing may occur. The region where sealing actually occurs—the actual sealing surface—may change within a given treatment session, from day to day, and from patient to patient, depending on a range of factors including for example, where the patient interface was placed on the face, tension in the positioning and stabilising structure and the shape of a patient's face.

In one form the target seal-forming region is located on an outside surface of the seal-forming structure 3100.

In certain forms of the present technology, the seal-forming structure 3100 is constructed from a biocompatible material, e.g. silicone rubber.

A seal-forming structure 3100 in accordance with the present technology may be constructed from a soft, flexible, resilient material such as silicone.

In certain forms of the present technology, a system is provided comprising more than one a seal-forming structure 3100, each being configured to correspond to a different size and/or shape range. For example the system may comprise one form of a seal-forming structure 3100 suitable for a large sized head, but not a small sized head and another suitable for a small sized head, but not a large sized head.

### 4.3.1.1 Sealing Mechanisms

In one form, the seal-forming structure includes a sealing flange utilizing a pressure assisted sealing mechanism. In use, the sealing flange can readily respond to a system positive pressure in the interior of the plenum chamber 3200 acting on its underside to urge it into tight sealing engagement with the face. The pressure assisted mechanism may act in conjunction with elastic tension in the positioning and stabilising structure.

In one form, the seal-forming structure 3100 comprises a sealing flange and a support flange. The sealing flange comprises a relatively thin member with a thickness of less than about 1 mm, for example about 0.25 mm to about 0.45 mm, which extends around the perimeter of the plenum chamber 3200. Support flange may be relatively thicker than the sealing flange. The support flange is disposed between the sealing flange and the marginal edge of the plenum chamber 3200, and extends at least part of the way around the perimeter. The support flange is or includes a spring-like element and functions to support the sealing flange from buckling in use.

In one form, the seal-forming structure may comprise a compression sealing portion or a gasket sealing portion. In use the compression sealing portion, or the gasket sealing portion is constructed and arranged to be in compression, e.g. as a result of elastic tension in the positioning and stabilising structure.

In one form, the seal-forming structure comprises a tension portion. In use, the tension portion is held in tension, e.g. by adjacent regions of the sealing flange.

In one form, the seal-forming structure comprises a region having a tacky or adhesive surface.

In certain forms of the present technology, a seal-forming structure may comprise one or more of a pressure-assisted sealing flange, a compression sealing portion, a gasket sealing portion, a tension portion, and a portion having a tacky or adhesive surface.

### 4.3.1.2 Nose Bridge or Nose Ridge Region

In one form, the non-invasive patient interface 3000 comprises a seal-forming structure that forms a seal in use on a nose bridge region or on a nose-ridge region of the patient's face.

In one form, the seal-forming structure includes a saddle-shaped region constructed to form a seal in use on a nose bridge region or on a nose-ridge region of the patient's face.

### 4.3.1.3 Upper Lip Region

In one form, the non-invasive patient interface 3000 comprises a seal-forming structure that forms a seal in use on an upper lip region (that is, the lip superior) of the patient's face.

In one form, the seal-forming structure includes a saddle-shaped region constructed to form a seal in use on an upper lip region of the patient's face.

### 4.3.1.4 Chin-Region

In one form the non-invasive patient interface 3000 comprises a seal-forming structure that forms a seal in use on a chin-region of the patient's face.

In one form, the seal-forming structure includes a saddle-shaped region constructed to form a seal in use on a chin-region of the patient's face.

### 4.3.1.5 Forehead Region

In one form, the seal-forming structure that forms a seal in use on a forehead region of the patient's face. In such a form, the plenum chamber may cover the eyes in use.

### 4.3.1.6 Nasal Pillows

In one form the seal-forming structure of the non-invasive patient interface 3000 comprises a pair of nasal puffs, or nasal pillows, each nasal puff or nasal pillow being constructed and arranged to form a seal with a respective naris of the nose of a patient.

Nasal pillows in accordance with an aspect of the present technology include: a frusto-cone, at least a portion of which forms a seal on an underside of the patient's nose, a stalk, a flexible region on the underside of the frusto-cone and connecting the frusto-cone to the stalk. In addition, the structure to which the nasal pillow of the present technology is connected includes a flexible region adjacent the base of the stalk. The flexible regions can act in concert to facilitate a universal joint structure that is accommodating of relative movement both displacement and angular of the frusto-cone and the structure to which the nasal pillow is connected. For example, the frusto-cone may be axially displaced towards the structure to which the stalk is connected.

### 4.3.1.7 Nose-Only Masks

In one form, the patient interface 3000 comprises a seal-forming structure 3100 configured to seal around an entrance to the patient's nasal airways but not around the patient's mouth. The seal-forming structure 3100 may be configured to seal to the patient's lip superior. The patient interface 3000 may leave the patient's mouth uncovered. This patient interface 3000 may deliver a supply of air or breathable gas to both nares of patient 1000 and not to the mouth. This type of patient interface may be identified as a nose-only mask.

One form of nose-only mask according to the present technology is what has traditionally been identified as a “nasal mask”, having a seal-forming structure 3100 configured to seal on the patient's face around the nose and over the bridge of the nose. A nasal mask may be generally triangular in shape. In one form, the non-invasive patient interface 3000 comprises a seal-forming structure 3100 that forms a seal in use to an upper lip region (e.g. the lip superior), to the patient's nose bridge or at least a portion of the nose ridge above the pronasale, and to the patient's face on each lateral side of the patient's nose, for example proximate the patient's nasolabial sulci. The patient interface 3000 shown in FIG. 3A has this type of seal-forming structure 3100. This patient interface 3000 may deliver a supply of air or breathable gas to both nares of patient 1000 through a single orifice.

Another form of nose-only mask may seal around an inferior periphery of the patient's nose without engaging the user's nasal ridge. This type of patient interface 3000 may be identified as a “nasal cradle” mask and the seal-forming structure 3100 may be identified as a “nasal cradle cushion”, for example. In one form, for example as shown in FIG. 3Z, the seal-forming structure 3100 is configured to form a seal in use with inferior surfaces of the nose around the nares. The seal-forming structure 3100 may be configured to seal around the patient's nares at an inferior periphery of the patient's nose including to an inferior and/or anterior surface of a pronasale region of the patient's nose and to the patient's nasal alae. The seal-forming structure 3100 may seal to the patient's lip superior. The shape of the seal-forming structure 3100 may be configured to match or closely follow the underside of the patient's nose and may not contact a nasal bridge region of the patient's nose or any portion of the patient's nose superior to the pronasale. In one form of nasal cradle cushion, the seal-forming structure 3100 comprises a bridge portion dividing the opening into two orifices, each of which, in use, supplies air or breathable gas to a respective one of the patient's nares. The bridge portion may be configured to contact or seal against the patient's columella in use. Alternatively, the seal-forming structure 3100 may comprise a single opening to provide a flow or air or breathable gas to both of the patient's nares.

In some forms, a nose-only mask may comprise nasal pillows, described above.

### 4.3.1.8 Nose and Mouth Masks

In one form, the patient interface 3000 comprises a seal-forming structure 3100 configured to seal around an entrance to the patient's nasal airways and also around the patient's mouth. The seal-forming structure 3100 may be configured to seal to the patient's face proximate a chin region. This patient interface 3000 may deliver a supply of air or breathable gas to both nares and to the mouth of patient 1000. This type of patient interface may be identified as a nose and mouth mask.

One form of nose-and-mouth mask according to the present technology is what has traditionally been identified as a “full-face mask”, having a seal-forming structure 3100 configured to seal on the patient's face around the nose, below the mouth and over the bridge of the nose. A nose-and-mouth mask may be generally triangular in shape. In one form the patient interface 3000 comprises a seal-forming structure 3100 that forms a seal in use to a patient's chin-region (which may include the patient's lip inferior and/or a region directly inferior to the lip inferior), to the patient's nose bridge or at least a portion of the nose ridge superior to the pronasale, and to cheek regions of the patient's face. The patient interface 3000 shown in FIG. 1C is of this type. This patient interface 3000 may deliver a supply of air or breathable gas to both nares and mouth of patient 1000 through a single orifice. This type of seal-forming structure 3100 may be referred to as a “nose-and-mouth cushion”.

In another form the patient interface 3000 comprises a seal-forming structure 3100 that forms a seal in use on a patient's chin region (which may include the patient's lip inferior and/or a region directly inferior to the lip inferior), to an inferior and/or an anterior surface of a pronasale portion of the patient's nose, to the alae of the patient's nose and to the patient's face on each lateral side of the patient's nose, for example proximate the nasolabial sulci. The seal-forming structure 3100 may also form a seal against a patient's lip superior. A patient interface 3000 having this type of seal-forming structure may have a single opening configured to deliver a flow of air or breathable gas to both nares and mouth of a patient, may have an oral hole configured to provide air or breathable gas to the mouth and a nasal hole configured to provide air or breathable gas to the nares, or may have an oral hole for delivering air to the patient's mouth and two nasal holes for delivering air to respective nares. This type of patient interface 3000 may have a nasal portion and an oral portion, the nasal portion sealing to the patient's face at similar locations to a nasal cradle mask.

In a further form of nose and mouth mask, the patient interface 3000 may comprise a seal-forming structure 3100 having a nasal portion comprising nasal pillows and an oral portion configured to form a seal to the patient's face around the patient's mouth.

In some forms, the seal-forming structure 3100 may have a nasal portion that is separate and distinct from an oral portion. In other forms, a seal-forming structure 3100 may form a contiguous seal around the patient's nose and mouth.

It is to be understood that the above examples of different forms of patient interface 3000 do not constitute an exhaustive list of possible configurations. In some forms a patient interface 3000 may comprise a combination of different features of the above described examples of nose-only and nose and mouth masks.

### 4.3.2 Plenum Chamber

The plenum chamber 3200 has a perimeter that is shaped to be complementary to the surface contour of the face of an average person in the region where a seal will form in use. In use, a marginal edge of the plenum chamber 3200 is positioned in close proximity to an adjacent surface of the face. Actual contact with the face is provided by the seal-forming structure 3100. The seal-forming structure 3100 may extend in use about the entire perimeter of the plenum chamber 3200. In some forms, the plenum chamber 3200 and the seal-forming structure 3100 are formed from a single homogeneous piece of material.

In certain forms of the present technology, the plenum chamber 3200 does not cover the eyes of the patient in use. In other words, the eyes are outside the pressurised volume defined by the plenum chamber. Such forms tend to be less obtrusive and/or more comfortable for the wearer, which can improve compliance with therapy.

In certain forms of the present technology, the plenum chamber 3200 is constructed from a transparent material, e.g. a transparent polycarbonate. The use of a transparent material can reduce the obtrusiveness of the patient interface, and help improve compliance with therapy. The use of a transparent material can aid a clinician to observe how the patient interface is located and functioning.

In certain forms of the present technology, the plenum chamber 3200 is constructed from a translucent material. The use of a translucent material can reduce the obtrusiveness of the patient interface, and help improve compliance with therapy.

In some forms, the plenum chamber 3200 is constructed from a rigid material such as polycarbonate. The rigid material may provide support to the seal-forming structure.

In some forms, the plenum chamber 3200 is constructed from a flexible material (e.g., constructed from a soft, flexible, resilient material like silicone, textile, foam, etc.). For example, in examples then may be formed from a material which has a Young's modulus of 0.4 GPa or lower, for example foam. In some forms of the technology the plenum chamber 3200 may be made from a material having Young's modulus of 0.1 GPa or lower, for example rubber. In other forms of the technology the plenum chamber 3200 may be made from a material having a Young's modulus of 0.7 MPa or less, for example between 0.7 MPa and 0.3 MPa. An example of such a material is silicone.

### 4.3.2.1 Multiple Openings

As shown in FIGS. 7A and 7B, different plenum chambers 3200-1, 3200-2 may be formed as part of a multi-opening cushion 3050-1, 3050-2. In the illustrated examples, the cushions 3050-1, 3050-2 each include three openings, although an alternate cushion may be formed with greater or fewer openings.

In some forms, the different openings may serve different functions. For example, some openings may be exclusively inlet openings, while other openings may be exclusively outlet openings.

In other forms, at least one opening may serve two different functions. For example, one opening may operate as both an inlet and an outlet during the same breathing cycle.

The plurality of openings may allow for a variety of configurations of air delivery to the plenum chamber 3200-1, 3200-2. For example, depending on patient need and/or patient comfort, the patient may use a given cushion 3050-1, 3050-2 in a “tube-up” configuration (e.g., using conduit headgear—described below) or a “tube-down” configuration (e.g., using a single conduit in front of the patient's face).

### 4.3.2.2 Nose and Mouth Mask

As shown in FIG. 7A, the plenum chamber 3200-1 includes a pair of plenum chamber inlet ports 3254-1, which may be used to convey gas into and/or out of the plenum chamber 3200-1. The plenum chamber inlet ports 3254-1 may be disposed on opposite sides (e.g., left and right sides) of the plenum chamber 3200-1.

In some forms, the plenum chamber 3200-1 may also include at least one vent opening 3402-1 (see e.g., FIG. 7A). The vent opening 3402-1 may be disposed in a center of the plenum chamber 3200-1. For example, the vent opening 3402-1 may be disposed between the plenum chamber inlet ports 3254-1.

In some forms, the plenum chamber 3200-1 may include a pair of grooves 3266-1. Each groove 3266-1 may be disposed proximate to one of the plenum chamber inlet ports 3254-1. Each groove 3266-1 may form a partially recessed surface.

### 4.3.2.3 Nose-Only Mask

The plenum chamber 3200-2 of a nasal only cushion 3050-2 may be similar to the plenum chamber 3200-1 of the mouth and nose cushion 3050-1. Only some similarities and differences between the plenum chambers 3200-1, 3200-2 may be described below.

As shown in FIG. 7B, the plenum chamber 3200-2 includes a pair of plenum chamber inlet ports 3254-2, which may be used to convey gas into and/or out of the plenum chamber 3200-2. The plenum chamber inlet ports 3254-2 may be disposed on opposite sides (e.g., left and right sides) of the plenum chamber 3200-2.

In some forms, the plenum chamber 3200-2 may also include at least one vent opening 3402-2 (see e.g., FIG. 7B). The vent opening 3402-2 may be disposed in a center of the plenum chamber 3200-2. For example, the vent opening 3402-2 may be disposed between the plenum chamber inlet ports 3254-2.

In some forms, the plenum chamber 3200-2 may include a pair of grooves 3266-2. Each groove 3266-2 may be disposed proximate to one of the plenum chamber inlet ports 3254-2. Each groove 3266-2 may form a partially recessed surface.

### 4.3.3 Positioning and Stabilising Structure

The seal-forming structure 3100 of the patient interface 3000 of the present technology may be held in sealing position in use by the positioning and stabilising structure 3300. The positioning and stabilising structure 3300 may comprise and function as “headgear” since it engages the patient's head in order to hold the patient interface 3000 in a sealing position. Examples of a positioning and stabilising structure may be shown in FIG. 3A.

In one form the positioning and stabilising structure 3300 provides a retention force at least sufficient to overcome the effect of the positive pressure in the plenum chamber 3200 to lift off the face.

In one form the positioning and stabilising structure 3300 provides a retention force to overcome the effect of the gravitational force on the patient interface 3000.

In one form the positioning and stabilising structure 3300 provides a retention force as a safety margin to overcome the potential effect of disrupting forces on the patient interface 3000, such as from tube drag, or accidental interference with the patient interface.

### 4.3.3.1 Conduit Headgear

### 4.3.3.1.1 Conduit Headgear Tubes

In some forms of the present technology, the positioning and stabilising structure 3300 comprises one or more headgear tubes 3350 that deliver pressurised air received from a conduit forming part of the air circuit 4170 from the RPT device to the patient's airways, for example through the plenum chamber 3200 and seal-forming structure 3100. In the form of the present technology illustrated in FIG. 3Z, the positioning and stabilising structure 3300 comprises two tubes 3350 that deliver air to the plenum chamber 3200 from the air circuit 4170. The tubes 3350 are configured to position and stabilise the seal-forming structure 3100 of the patient interface 3000 at the appropriate part of the patient's face (for example, the nose and/or mouth) in use. This allows the conduit of air circuit 4170 providing the flow of pressurised air to connect to a connection port 3600 of the patient interface in a position other than in front of the patient's face, for example on top of the patient's head.

In the form of the present technology illustrated in FIG. 3Z, the positioning and stabilising structure 3300 comprises two tubes 3350, each tube 3350 being positioned in use on a different side of the patient's head and extending across the respective cheek region, above the respective ear (superior to the otobasion superior on the patient's head) to the elbow 3610 on top of the head of the patient 1000. This form of technology may be advantageous because, if a patient sleeps with their head on its side and one of the tubes 3350 is compressed to block or partially block the flow of gas along the tube 3350, the other tube 3350 remains open to supply pressurised gas to the patient. In other examples of the technology, the patient interface 3000 may comprise a different number of tubes, for example one tube, or two or more tubes.

In one example in which the patient interface has one tube 3350, the single tube 3350 is positioned on one side of the patient's head in use (e.g. across one cheek region) and a strap forms part of the positioning and stabilising structure 3300 and is positioned on the other side of the patient's head in use (e.g. across the other region) to assist in securing the patient interface 3000 on the patient's head. For example, the tube 3350 and the strap may each be under tension in use in order to assist in maintaining the seal-forming structure 3100 in a sealing position.

In one form, the tube 3350 may be at least partially extensible so that the tube 3350 and the strap may adjust substantially equal lengths when worn by a patient. This may allow for substantially symmetrical adjustments between the tube 3350 and the strap so that the seal-forming structure remains substantially in the middle.

In the form of the technology shown in FIG. 3Z-1, the two tubes 3350 are fluidly connected at superior ends to each other and to the connection port 3600. In some examples, the two tubes 3350 are integrally formed while in other examples the tubes 3350 are formed separately but are connected in use and may be disconnected, for example for cleaning or storage. Where separate tubes are used, they may be indirectly connected together, for example each may be connected to a T-shaped connector. The T-shaped connector may have two arms/branches each fluidly connectable to a respective one of the tubes 3350. Additionally, the T-shaped connector may have a third arm or opening providing the connection port 3600 for fluid connection to the air circuit 4170 in use. The opening may be an inlet 3332 (see e.g., 7C) for receiving the flow of pressurized air.

In some forms, a Y-shaped connector may be used instead of the T-shaped connector. The first two arms may be oblique with respect to one another, and the third arm may be oblique with respect to the first two arms. The angled formation of the first two arms may be similar to the shape of the patient's head in order to conform to the shape.

In some forms, at least one of the arms of the T-shaped connector (or Y-shaped connector) may be flexible. This may allow the connector to bend based on the shape of the patient's head and/or a force in the positioning and stabilising structure 3300.

In some forms, at least one of the arms of the T-shaped connector (or Y-shaped connector) may be at least partially rigidised. This may assist in maintaining the shape of the connector so that bending of the connector does not close the airflow path.

In certain forms of the present technology the patient interface 3000 is configured such that the connection port 3600 can be positioned in a range of positions across the top of the patient's head so that the patient interface 3000 can be positioned as appropriate for the comfort or fit of an individual patient. In some examples, the headgear tubes 3350 are configured to allow movement of an upper portion of the patient interface 3000 (e.g. a connection port 3600) with respect to a lower portion of the patient interface 3000 (e.g. a plenum chamber 3200). That is, the connection port 3600 may be at least partially decoupled from the plenum chamber 3200. In this way, the seal-forming structure 3100 may form an effective seal with the patient's face irrespective of the position of the connection port 3600 (at least within a predetermined range of positions) on the patient's head.

As described above, in some examples of the present technology the patient interface 3000 comprises a seal-forming structure 3100 in the form of a cradle cushion which lies generally under the nose and seals to an inferior periphery of the nose (e.g. an under-the-nose cushion). The positioning and stabilising structure 3300, including the tubes 3350 may be structured and arranged to pull the seal-forming structure 3100 into the patient's face under the nose with a sealing force in a posterior and superior direction (e.g. a posterosuperior direction). A sealing force with a posterosuperior direction may cause the seal-forming structure 3100 to form a good seal to both the inferior periphery of the patient's nose and anterior-facing surfaces of the patient's face, for example on either side of the patient's nose and the patient's lip superior.

### 4.3.3.1.2 Conduit Headgear Connection Port

In certain forms of the present technology, the patient interface 3000 may comprise a connection port 3600 located proximal to a superior, lateral or posterior portion of a patient's head. For example, in the form of the present technology illustrated in FIG. 3Z-1, the connection port 3600 is located on top of the patient's head (e.g. at a superior location with respect to the patient's head). In this example the patient interface 3000 comprises an elbow 3610 forming the connection port 3600. The elbow 3610 may be configured to fluidly connect with a conduit of an air circuit 4170. The elbow 3610 may be configured to swivel with respect to the positioning and stabilising structure 3300 to at least partially decouple the conduit from the positioning and stabilising structure 3300. In some examples the elbow 3610 may be configured to swivel by rotation about a substantially vertical axis and, in some particular examples, by rotation about two or more axes. In some examples the elbow may comprise or be connected to the tubes 3350 by a ball-and-socket joint. The connection portion 3600 may be located in the sagittal plane of the patient's head in use.

### 4.3.3.1.3 Headgear Tube Fluid Connections

The two tubes 3350 are fluidly connected at their inferior ends to the plenum chamber 3200. In certain forms of the technology, the connection between the tubes 3350 and the plenum chamber 3200 is achieved by connection of two rigid connectors. The tubes 3350 and plenum chamber 3200 may be configured to enable the patient to easily connect the two components together in a reliable manner. The tubes 3350 and plenum chamber 3200 may be configured to provide tactile and/or audible feedback in the form of a ‘re-assuring click’ or a similar sound, so that the patient may easily know that each tube 3350 has been correctly connected to the plenum chamber 3200. In one form, the tubes 3350 are formed from a silicone or textile material and the inferior end of each of the silicone tubes 3350 is overmolded to a rigid connector made, for example, from polypropylene, polycarbonate, nylon or the like. The rigid connector on each tube 3350 may comprise a female mating feature configured to connect with a male mating feature on the plenum chamber 3200. Alternatively, the rigid connector on each tube 3350 may comprise a male mating feature configured to connect to a female mating feature on the plenum chamber 3200. In other examples the tubes 3350 may each comprise a male or female connector formed from a flexible material, such as silicone or TPE, for example the same material from which the tubes 3350 are formed.

In other examples a compression seal is used to connect each tube 3350 to the plenum chamber 3200. For example, a resiliently flexible (e.g. silicone) tube 3350 without a rigid connector may be configured to be squeezed to reduce its diameter so that it can be compressed into a port in the plenum chamber 3200 and the inherent resilience of the silicone pushes the tube 3350 outwards to seal the tube 3350 in the port in an air-tight manner. Alternatively, in a hard-to-hard type engagement between the tube 3350 and the plenum chamber 3200, each tube 3350 and/or plenum chamber 3200 may comprise a pressure activated seal, for example a peripheral sealing flange. When pressurised gas is supplied through the tubes 3350 the sealing flange may be urged against the join between the tubes and a circumferential surface around a port or connector of the plenum chamber 3200 to form or enhance a seal between the tube 3350 and plenum chamber 3200.

### 4.3.3.2 Headgear Straps

In some forms, the positioning and stabilising structure 3300 may include headgear 3302 with at least one strap which may be worn by the patient in order to assist in properly orienting the seal-forming structure 3100 against the patient's face (e.g., in order to limit or prevent leaks).

As described above, some forms of the headgear 3302 may be constructed from a textile material, which may be comfortable against the patient's skin. The textile may be flexible in order to conform to a variety of facial contours. Although the textile may include rigidisers along a selected length, which may limit bending, flexing, and/or stretching of the headgear 3302.

In certain forms, the headgear 3302 may be at least partially extensible. For example, the headgear 3302 may include elastic, or a similar extensible material. For example, the entire headgear 3302 may be extensible or selected portions may be extensible (or more extensible than surrounding portions). This may allow the headgear 3302 to stretch while under tension, which may assist in providing a sealing force for the seal-forming structure 3100.

In certain forms of the present technology, the positioning and stabilising structure 3300 comprises at least one headgear strap acting in addition to the tubes 3350 to position and stabilise the seal-forming structure 3100 at the entrance to the patient's airways. As shown in FIG. 3Z-1, the patient interface 3000 comprises a strap 3307-2 forming part of the positioning and stabilising structure 3300. The strap 3307-2 may be known as a back strap or a rear headgear strap, for example. The rear strap 3307-2 may overlay the temporal bones, parietal bone, and/or occipital bone. In other examples of the present technology, one or more further straps may be provided. For example, patient interfaces 3000 according to examples of the present technology having a nose-and-mouth cushion may have a second, lower, strap configured to lie against the patient's head proximate the patient's neck and/or against posterior surfaces of the patient's neck.

In the example shown in FIG. 3Z-1, strap 3310 of the positioning and stabilising structure 3300 is connected between the two tubes 3350 positioned on each side of the patient's head and passing around the back of the patient's head, for example overlying or lying inferior to the occipital bone of the patient's head in use. The strap 3310 connects to each tube above the patient's ears. With reference to FIG. 3Z-1, the positioning and stabilising structure 3300 comprises a pair of tabs 3320. In use a strap 3310 may be connected between the tabs 3320. The strap 3310 may be sufficiently flexible to pass around the back of the patient's head and lie comfortably against the patient's head, even when under tension in use.

### 4.3.4 Vent

In one form, the patient interface 3000 includes a vent 3400 constructed and arranged to allow for the washout of exhaled gases, e.g. carbon dioxide.

In certain forms the vent 3400 is configured to allow a continuous vent flow from an interior of the plenum chamber 3200 to ambient whilst the pressure within the plenum chamber is positive with respect to ambient. The vent 3400 is configured such that the vent flow rate has a magnitude sufficient to reduce rebreathing of exhaled CO2 by the patient while maintaining the therapeutic pressure in the plenum chamber in use.

One form of vent 3400 in accordance with the present technology comprises a plurality of holes, for example, about 20 to about 80 holes, or about 40 to about 60 holes, or about 45 to about 55 holes.

The vent 3400 may be located in the plenum chamber 3200. Alternatively, the vent 3400 is located in a decoupling structure, e.g., a swivel, or connected to the end of a short tube 3290 as illustrated in FIG. 8, optionally via an HME.

As shown in FIG. 7N, a vent 3450 may be used with the patient interface 3000. The vent 3450 may have a substantially similar shape to the vent opening 3402-1 (e.g., a substantially circular shape).

The vent 3450 may be used with either the mouth and nose plenum chamber 3200-1 (e.g., illustrated in FIGS. 7A) or the nose-only plenum chamber 3200-2 (e.g., illustrated in FIGS. 7B and 8).

With continued reference to FIG. 7A, the vent 3450 may include a vent housing 3404, which may be configured to engage with the vent opening 3402. The vent housing 3404 may be constructed from a rigid material or a semi-rigid material. For example, the vent housing 3404 may be constructed from plastic, metal, or any similar material. The vent housing 3404 may add rigidity to the patient interface 3000 (e.g., to limit unwanted bending that may affect the position of the seal-forming structure 3100 on the patient's face). The vent housing 3404 may include an anterior surface 3408, a posterior surface 3412, and a groove 3416. The anterior surface 3408 faces away from the patient's face in use, and may be positioned outside the pressurized volume of the plenum chamber 3200. The posterior surface 3412 is disposed opposite to the anterior surface 3408. In use, the posterior surface 3412 may face the patient and may be disposed within the pressurized volume of the plenum chamber 3200. The groove 3416 may be formed between the anterior and posterior surfaces 3408, 3412. A portion of the plenum chamber 3200 may be received within the groove 3416 in order to retain the vent 3400 in position.

In some forms, a diffuser 3448 may be used with the vent housing 3404. The diffuser 3448 may assist with limiting the decibel output from any of the patient interface 3000 (or any other patient interface). Specifically, the diffuser 3448 may assist in limiting the decibel level associated with air output from the patient interface 3000 (e.g., exhaled air), although the diffuser 3448 may limit the decibel level of at any point in the patient interface.

In certain forms, the diffuser 3448 may diffuse, and therefore slow, the exhaust gas exiting the plenum chamber 3200 and passing through the vent housing 3404. The diffuser 3448 may assist in avoiding jetting and associated discomfort to the patient and/or bed partner (e.g., noise caused by jetting against a pillow, sheets, bedclothes, etc.).

In some forms, the diffuser may include an anterior surface 3456 that faces away from the patient in use. An outer diameter of the anterior surface 3456 may be less than an inner diameter of the vent housing 3404 proximate to the anterior surface 3408. This may form a gap 3464 through which air may travel.

### 4.3.5 Decoupling Structure(s)

In one form the patient interface 3000 includes at least one decoupling structure, for example, a swivel or a ball and socket.

### 4.3.6 Connection Port

Connection port 3600 allows for connection to the air circuit 4170.

### 4.3.7 Forehead Support

In one form, the patient interface 3000 includes a forehead support 3700.

### 4.3.8 Anti-Asphyxia Valve

In one form, the patient interface 3000 includes an anti-asphyxia valve.

### 4.3.9 Ports

In one form of the present technology, a patient interface 3000 includes one or more ports that allow access to the volume within the plenum chamber 3200. In one form this allows a clinician to supply supplementary oxygen. In one form, this allows for the direct measurement of a property of gases within the plenum chamber 3200, such as the pressure.

### 4.3.10 Heat and Moisture Exchanger (HME)

In forms of the technology a heat and moisture exchanger (HME) 5300 may be formed from a heat and moisture exchange component 5301 and optionally, retaining structure 5320. An alternative HMEs 5300-1-4 exemplified comprise a heat and moisture exchange component 5301, optional retaining structure 5320 and a thermally conductive member 5330.

An HME may be located in a range of different locations within a patent interface, such that incoming air flow to the patient passes through the HME, contacting the heat and moisture exchange material before inhalation, and at least a portion of the gases exhaled from the patient pass through the HME in an opposing direction to the incoming air flow, before being vented or partially vented to ambient, or being returned to the air circuit. In examples, an HME may be located within the plenum chamber 3200 or externally to the plenum chamber, in connection with a tube of air circuit 4170, via vent opening 3402, for example as seen in FIG. 8.

FIG. 8. shows an HME 5300 or 5300-1 adapted for use in a nasal mask in one configuration. HME 5300/5300-1 is received within vent opening 3402, between plenum chamber 3200 and vent housing 3404. In use, incoming air flow generated by the flow generator enters HME 5300/5300-1, passes through and enters plenum chamber 3200 via short tube 3290. On patient exhalation, gases leave plenum chamber 3200, pass through short tube 3290, HME 5300/5300-1 and at least partially exit to ambient through vent 3400. HME 5300/5301 may be removably received by HME housing 5350.

Use of HME 5300 without a thermally conductive member, within an RPT device is demonstrated in FIG. 9 that shows inhalation through the HME and FIG. 10 showing exhalation of patient gases through the HME in the opposite direction. During inhalation, air 5401 enters the HME 5300 on flow generator side 5310 of the HME distal from the plenum chamber, passes through heat and moisture exchange material 5301 and exits 5402 material 5301 on patient side 5311 of the HME, proximal to the plenum chamber. Air entering 5401 originating from the flow generator may be in the range of 25-27° C.

On exhalation from the patient, air will likely be warmer, in the range of 34-35° C. Exhaled air 5403 leaves patient and enters patient-facing side 5311 of material 5301, depositing moisture 5410 on material 5301. A portion of air exiting material 5301 may return 5404 to the air circuit 4170, and a portion may be vented to ambient 5405.

Use of HME 5300-1 to humidify air within an RPT device is demonstrated in FIGS. 19-21, in contrast with the air movement using HME 5300. In the example shown, HME 5300-1 comprises heat and moisture exchange component 5301, and thermally conductive member 5330, in the form of a cover mounted on the patient side 5311 of heat and moisture exchange component 5301.

FIG. 19 shows the initial inhalation of air by a patient. Air supplied by RPT device 4000 via air circuit 4170 provides an incoming air supply 6000 to the patient via patient interface 3000. Incoming air 6000 enters HME 5300-1, passing through dry heat and moisture exchange material 5301 and thermally conductive member 5330 into plenum chamber 3200 for inhalation by patient. As air passes through HME 5300-1, the temperature of the HME is brought closer to air supply temperature.

FIG. 20 shows the movement of gases following patient exhalation. Exhaled gases (usually above ambient temperature and humidity) 6010 pass through the HME 5300-1 and contacts thermally conductive member 5330, transferring an amount of heat from the exhaled gases to member 5330. The reduction of heat of exhaled air 6010 facilitates deposition of moisture 6005 from exhaled air 6010 onto heat and moisture exchange component 5301. Exhaled air returns to one or more of air circuit 4170 (6020) or is vented to ambient 6030 via vent structures in the patient interface.

FIG. 21 shows the movement of air during a secondary inhalation. Air 6000-1 is supplied to the patient via patient interface 3000 by RPT device 4000 via air circuit 4170. Air 6000-1 passes through heat and moisture exchange component 5301 that includes moisture 6005 deposited during exhalation. Temperature of air 6000-1 is to increases as a result of increased temperature of the thermally conductive member 5330, raising the saturation point of air 6000-1 and facilitating uptake of moisture into air 6000-1. Humidified air 6007 then exits HME 5300-1 into plenum chamber 3200 for inhalation by the patient.

In the present technology, a thermally conductive member is incorporated in use with the heat and moisture exchange component, forming HME 5300-1 as seen in FIGS. 11-21.

Heat and moisture exchangers may include a heat and moisture exchange component 5301 which may comprise for example, a foam or a non-woven material.

Examples of suitable heat and moisture exchange materials include HME7 foam, made by FoamPartner, paper or card. In some examples, such as that shown in FIG. 8A, the heat and moisture exchange component comprises a cylindrical, permeable three-dimensional structure, with opposing faces separated by a continuous wall. The cylindrical shape has a core structure formed from the heat and moisture exchange material, the structure may include perforations, channels, porous regions and/or apertures aligned in the directions of air and gas flow through the heat and moisture exchange component, while providing surface area for moisture deposition.

As exemplified in FIG. 8A, heat and moisture exchange component 5301 may be formed to include a three-dimensional honeycomb structure 5302, formed from an array of hollow tubes or cells made from a heat and moisture exchange material, the open ends of the hollow tubes or cells aligned to be substantially perpendicular to the flow of gases through the HME, each tube or cell defined by walls such that gases freely flow through the cylindrical heat and moisture exchange component 5301 during both inhalation and exhalation, with minimal impedance. These walls provide surface area for moisture deposition as the patient exhales, and surface area for dry air to cross during inhalation, picking up the moisture as the air moves through the material. The surface areas may be increased or decreased by changing the density of the hollow tube or cells within the heat and moisture exchange material.

Heat and moisture exchange component 5301 may be surrounded or partially surrounded by a retaining structure 5320. Retaining structure 5320 may support heat and moisture exchange component 5301 in any required shape, provide structure and/or strength and aid in connection to components of the patient interface or an HME housing, for example via clips, threads, flanges, interlocking components or friction fit components. The retaining structure may be integrally formed with the heat and moisture exchange material in some examples. In FIG. 8A, retaining structure 5320 is in the form of a hollow cylinder, supporting heat and moisture exchange component as a solid wall surrounding the honeycomb structure 5302, which assists in ensuring correct alignment of the hollow structures to optimize air flow through the heat and moisture component 5301.

In further forms of the technology as shown in FIGS. 11-16, HME 5300-1 includes thermally conductive member 5330 in contact with heat and moisture exchange component (FIG. 8A) 5301 and/or retaining structure 5320. Placement of thermally conductive member 5330 between the heat and moisture exchange component and the patient may act to reduce the temperature of the exhaled breath of the user as it contacts the thermally conductive member 5330, and increase the amount of water which condenses onto, and is captured by the heat and moisture exchange material within component 5301.

Thermally conductive material 5330 may be formed from a material having a thermal conductivity (k) of between 10 W/m·K-1000 W/m·K. In some forms of the technology the thermal conductivity of the thermally conductive member is between 50 and 500 W/m·K. Metals are particularly suitable for use in forming, or being incorporated within the thermally conductive member, due to their higher thermal conductivity compared to materials utilised for the heat and moisture exchange material. A list of common thermal conductivities is provided in Table 2 as examples of possible materials for use as/including in thermally conductive members:

TABLE 2
Estimated Thermal Conductivity (k) W/(mK) at 20° C.

| Material | Thermal conductivity (W/(mK)) |
|---|---|
| Plastic | 0.23 |
| Paper | 0.03 |
| Wool | 0.04 |
| Polyurethane Foam | 0.03 |
| Aluminium | 237 |
| Aluminium nitride | 321 |
| Copper | 401 |
| Silver | 429 |
| Gold | 314 |
| Silicon carbide | 120 |
| Steel (1% carbon) | 43 |
| Stainless Steel | 16 |
| Brass | 109 |
| Tungsten | 175 |
| Zinc | 116 |

In some examples of the present invention, the thermally conductive member may be in the form of one or more of; a flat or contoured metal sheet(s), a foil sheet, mesh, wire mesh, perforated metal sheeting, casing or covering (FIGS. 11-15) wire (FIG. 16) or plate. The thermally conductive member may be formed entirely or partially of a thermally conductive material, and may be a formed from one or a combination of different thermally conductive materials. In further examples the thermally conductive member may include non, or lower thermally conductive materials in combination with higher thermally conductive materials.

In the example shown in FIG. 11, heat and moisture exchange component 5301 has a substantially cylindrical shape having a flow generator side 5310 and an opposing patient facing side 5311, separated circumferentially by wall 5312, and optionally supported around wall 5312 by retaining structure 5320. Thermally conductive member 5330 is formed from a thermally conductive material and shaped as a cover to receive a portion of heat and moisture exchange component 5301. Thermally conductive member 5330 comprises a substantially circular face plate 5331 and side wall 5332 extending substantially perpendicularly in a first direction around the circumference of face plate 5331. Face plate 5331 may include a plurality of venting structures, for example in the form of apertures 5336 to reduce impedance and enable air flow through the HME 5300-1.

In further forms of the invention, venting structures of the thermally conductive member may include, but are not limited to apertures, slots, areas of different thickness, diffusers or gaps arranged randomly or uniformly.

In examples shown in FIGS. 11-13, side wall 5332 may be shaped to extend from face plate 5331 partially along and radially outside wall 5312 of heat and moisture exchange component 5301 to form a cover over the patient facing side 5311 of the heat and moisture exchange component. In alternative examples of HME 5300-2 as seen in FIGS. 14 and 15, side wall 5332 may extend to cover the majority of the wall 5312 of heat and moisture exchange component 5301, acting as a casing or surround rather than a cover for one side of the heat and moisture exchange component 5301.

Heat and moisture exchange component 5301 may be adapted to be removably received within the cover or casing using a press fit, friction fit, clips, clasps or other connection means. This enables replacement of a worn heat and moisture exchange component or enables the user to add moisture to the heat and moisture exchange material to increase the availability of moisture within the patient interface.

In other forms not shown in the figures, the HME may comprise one or more of a covering structure, flange, surround, casing, receptacle or retaining structure formed from, partially formed from, or comprising the thermally conductive member.

FIG. 16 provides an example of an HME 5300-3 comprising heat and moisture exchange component 5301, with thermally conductive member in the form of a wire 5330-2, or multiple wires mounted on the patient side 5311 of heat and moisture exchange component 5301. Wire 5330-2 may be secured to a retaining structure 5320 at its terminal ends 5321, with the wire length mounted to form a winding path of thermally conductive material extending across one or more surfaces of heat and moisture exchange component 5301 multiple times, including internal and external walls or faces. Wire 5330-2 me be wound in the form of a spiral, of in a zig-zag formation as shown in FIG. 16 for example. The surface area and therefore heat transfer capacity may be increased by increasing the density of the winding path, length, thickness, cross-sectional shape or number of wire(s) 5330-2 or part thereof, and decreased by reducing these parameters, resulting in a decreased surface area. Wire 5330-2 may abut an outer surface of heat and moisture exchange component 5301, or may be embedded or partially embedded within the heat and moisture exchange component 5301 and/or retaining structure 5302 (not shown). In other examples wire(s) 5330-2 may extend outwardly from the perimeter of heat and moisture exchange component 5301 in any direction.

In still other examples, two of more materials with different thermally conductive properties may be used in a single HME to impart differing thermally conductivities to the HME at different regions, for example to create a temperature gradient.

FIGS. 17 and 18 show examples of a further form of the present invention HME 5300-4 for use in a patient interface wherein the thermally conductive member of the HME 5300-4 is at least in partial contact with ambient air. Over time, HMEs positioned within a patient interface can reach a steady-state temperature, reducing the ability of the HME to retain moisture and therefore effectively humidify the air flow to the patient. Enabling contact between the thermally conductive member and ambient air may act to cool the thermally conductive member, transferring heat away from the HME to ambient and therefore improving the moisture retention capabilities of the HME.

Projection structure(s) 5337 comprises part of the HME and is adapted for connection to an HME located within or partially within (for example) vent opening 3402-1 of plenum chamber 3200-1 of a patient interface. Thermally conductive member 5330 covers or partially covers the patient side 5311 of the heat and moisture exchange component 5301. Projection structure 5337 may be formed from the same or different thermally conductive material as thermally conductive member 5330 and is thermally connected to thermally conductive member 5330. Projection structure 5337 and thermally conductive member 5330 may be integrally formed or may be separate components removably connectable together.

In one example, seen in FIG. 17, projection structure 5337 is separable from thermally conductive member 5330 and removably connects directly or indirectly to wall 5332 of thermally conductive member 5330, distal from face plate 5331, via a central aperture (not shown). When projection structure 5337 is connected, air circuit 4170 is connectable to vent opening 3402-1 (optionally via an elbow), through central aperture of projection structure 5337, and projection structure 5337 or a portion thereof, is positioned against or on a portion of the anterior face of the plenum chamber 3000.

Projection structure 5337 comprises flange portion(s) 5338 shaped to abut the anterior face of plenum chamber 3200-1, connecting thermally conductive member 5330 with ambient air. In the example of FIG. 17, flange portion(s) 5338 surrounds vent opening 3402-1 and comprises two connected, curved wing regions on opposing sides of central aperture 5339, the wing regions curved to correspond to the anterior face of plenum chamber 3200-1. In other examples not shown flange portion(s) 5338 may be of varying sizes and shapes to correspond to contours on the anterior surface of the plenum chamber and may be curved or flat with respect to the anterior surface of the plenum chamber, and may include one or multiple flanges. Larger projection structures 5337 provide a larger surface area for heat transfer to ambient, potentially increasing the effectiveness of the HME 5300-4 over longer periods of time.

Alternatively, projection structure 5337 may be integrally formed with the thermally conductive member of the HME 5300-4 as seen in FIG. 18 and provide a surface for contact with ambient air externally to the plenum chamber.

Projection structures 5337 may include addition features such as textured surfaces, raised or depressed features, grooves 5340 or ribs for example to increase the surface area of projection structure 5337. The design of projection structure 5337 may be tailored to the level of humidity and heat required within the patient interface.

### 4.4 Humidifier

### 4.4.1 Humidifier Overview

In one form of the present technology there is provided a humidifier 5000 (e.g. as shown in FIG. 5A) to change the absolute humidity of air or gas for delivery to a patient relative to ambient air. Typically, the humidifier 5000 is used to increase the absolute humidity and increase the temperature of the flow of air (relative to ambient air) before delivery to the patient's airways.

The humidifier 5000 may comprise a humidifier reservoir 5110, a humidifier inlet 5002 to receive a flow of air, and a humidifier outlet 5004 to deliver a humidified flow of air. In some forms, as shown in FIG. 5A and FIG. 5B, an inlet and an outlet of the humidifier reservoir 5110 may be the humidifier inlet 5002 and the humidifier outlet 5004 respectively. The humidifier 5000 may further comprise a humidifier base 5006, which may be adapted to receive the humidifier reservoir 5110 and comprise a heating element 5240.

### 4.5 Breathing Waveforms

FIG. 6A shows a model typical breath waveform of a person while sleeping. The horizontal axis is time, and the vertical axis is respiratory flow rate. While the parameter values may vary, a typical breath may have the following approximate values: tidal volume Vt 0.5 L, inhalation time Ti 1.6 s, peak inspiratory flow rate Qpeak 0.4 L/s, exhalation time Te 2.4 s, peak expiratory flow rate Qpeak −0.5 L/s. The total duration of the breath, Ttot, is about 4 s. The person typically breathes at a rate of about 15 breaths per minute (BPM), with Ventilation Vent about 7.5 L/min. A typical duty cycle, the ratio of Ti to Ttot, is about 40%.

### 5 GLOSSARY

For the purposes of the present technology disclosure, in certain forms of the present technology, one or more of the following definitions may apply. In other forms of the present technology, alternative definitions may apply.

### 5.1 General

Air: In certain forms of the present technology, air may be taken to mean atmospheric air, and in other forms of the present technology air may be taken to mean some other combination of breathable gases, e.g. oxygen enriched air.

Ambient: In certain forms of the present technology, the term ambient will be taken to mean (i) external of the treatment system or patient, and (ii) immediately surrounding the treatment system or patient.

For example, ambient humidity with respect to a humidifier may be the humidity of air immediately surrounding the humidifier, e.g. the humidity in the room where a patient is sleeping. Such ambient humidity may be different to the humidity outside the room where a patient is sleeping.

In another example, ambient pressure may be the pressure immediately surrounding or external to the body.

In certain forms, ambient (e.g., acoustic) noise may be considered to be the background noise level in the room where a patient is located, other than for example, noise generated by an RPT device or emanating from a mask or patient interface. Ambient noise may be generated by sources outside the room.

Automatic Positive Airway Pressure (APAP) therapy: CPAP therapy in which the treatment pressure is automatically adjustable, e.g. from breath to breath, between minimum and maximum limits, depending on the presence or absence of indications of SDB events.

Continuous Positive Airway Pressure (CPAP) therapy: Respiratory pressure therapy in which the treatment pressure is approximately constant through a respiratory cycle of a patient. In some forms, the pressure at the entrance to the airways will be slightly higher during exhalation, and slightly lower during inhalation. In some forms, the pressure will vary between different respiratory cycles of the patient, for example, being increased in response to detection of indications of partial upper airway obstruction, and decreased in the absence of indications of partial upper airway obstruction.

Flow rate: The volume (or mass) of air delivered per unit time. Flow rate may refer to an instantaneous quantity. In some cases, a reference to flow rate will be a reference to a scalar quantity, namely a quantity having magnitude only. In other cases, a reference to flow rate will be a reference to a vector quantity, namely a quantity having both magnitude and direction. Flow rate may be given the symbol Q. ‘Flow rate’ is sometimes shortened to simply ‘flow’ or ‘airflow’.

In the example of patient respiration, a flow rate may be nominally positive for the inspiratory portion of a breathing cycle of a patient, and hence negative for the expiratory portion of the breathing cycle of a patient. Device flow rate, Qd, is the flow rate of air leaving the RPT device. Total flow rate, Qt, is the flow rate of air and any supplementary gas reaching the patient interface via the air circuit. Vent flow rate, Qv, is the flow rate of air leaving a vent to allow washout of exhaled gases. Leak flow rate, Ql, is the flow rate of leak from a patient interface system or elsewhere. Respiratory flow rate, Qr, is the flow rate of air that is received into the patient's respiratory system.

Flow therapy: Respiratory therapy comprising the delivery of a flow of air to an entrance to the airways at a controlled flow rate referred to as the treatment flow rate that is typically positive throughout the patient's breathing cycle.

Humidifier: The word humidifier will be taken to mean a humidifying apparatus constructed and arranged, or configured with a physical structure to be capable of providing a therapeutically beneficial amount of water (H₂O) vapour to a flow of air to ameliorate a medical respiratory condition of a patient.

Leak: The word leak will be taken to be an unintended flow of air. In one example, leak may occur as the result of an incomplete seal between a mask and a patient's face. In another example leak may occur in a swivel elbow to the ambient.

Noise, conducted (acoustic): Conducted noise in the present document refers to noise which is carried to the patient by the pneumatic path, such as the air circuit and the patient interface as well as the air therein. In one form, conducted noise may be quantified by measuring sound pressure levels at the end of an air circuit.

Noise, radiated (acoustic): Radiated noise in the present document refers to noise which is carried to the patient by the ambient air. In one form, radiated noise may be quantified by measuring sound power/pressure levels of the object in question according to ISO 3744.

Noise, vent (acoustic): Vent noise in the present document refers to noise which is generated by the flow of air through any vents such as vent holes of the patient interface.

Oxygen enriched air: Air with a concentration of oxygen greater than that of atmospheric air (21%), for example at least about 50% oxygen, at least about 60% oxygen, at least about 70% oxygen, at least about 80% oxygen, at least about 90% oxygen, at least about 95% oxygen, at least about 98% oxygen, or at least about 99% oxygen. “Oxygen enriched air” is sometimes shortened to “oxygen”.

Medical Oxygen: Medical oxygen is defined as oxygen enriched air with an oxygen concentration of 80% or greater.

Patient: A person, whether or not they are suffering from a respiratory condition.

Pressure: Force per unit area. Pressure may be expressed in a range of units, including cmH₂O, g-f/cm² and hectopascal. 1 cmH₂O is equal to 1 g-f/cm² and is approximately 0.98 hectopascal (1 hectopascal=100 Pa=100 N/m²=1 millibar˜0.001 atm). In this specification, unless otherwise stated, pressure is given in units of cmH2O.

The pressure in the patient interface is given the symbol Pm, while the treatment pressure, which represents a target value to be achieved by the interface pressure Pm at the current instant of time, is given the symbol Pt.

Respiratory Pressure Therapy: The application of a supply of air to an entrance to the airways at a treatment pressure that is typically positive with respect to atmosphere.

Ventilator: A mechanical device that provides pressure support to a patient to perform some or all of the work of breathing.

### 5.2 Materials & their Properties

Hardness: Refers to durometer or indentation hardness, which is a material property measured by indentation of an indentor (e.g., as measured in accordance with ASTM D2240).

‘Soft’ materials may include silicone or thermo-plastic elastomer (TPE), and may, e.g. readily deform under finger pressure.

‘Hard’ materials may include polycarbonate, polypropylene, and may not e.g. readily deform under finger pressure.

Silicone or Silicone Elastomer: A synthetic rubber. In this specification, a reference to silicone is a reference to liquid silicone rubber (LSR) or a compression moulded silicone rubber (CMSR). One form of commercially available LSR is SILASTIC (included in the range of products sold under this trademark), manufactured by Dow Corning. Another manufacturer of LSR is Wacker. Unless otherwise specified to the contrary, an exemplary form of LSR has a Shore A (or Type A) indentation hardness in the range of about 35 to about 45 as measured using ASTM D2240.

### 5.3 Axes

Neutral axis: An axis in the cross-section of a beam or plate along which there are no longitudinal stresses or strains.

Longitudinal axis: An axis extending along the length of a shape. The axis generally passes through a center of the shape.

Circumferential axis: An axis oriented perpendicularly with respect to the longitudinal axis. The axis may be specifically present in pipes, tubes, cylinders, or similar shapes with a circular and/or elliptical cross section.

Deformation: The process where the original geometry of a member changes when subjected to forces, e.g. a force in a direction with respect to an axis. The process may include stretching or compressing, bending and, twisting.

Elasticity: The ability of a material to return to its original geometry after deformation.

Floppy structure or component: A structure or component that will change shape, e.g. bend, when caused to support its own weight, within a relatively short period of time such as 1 second.

Resilience: Ability of a material to absorb energy when deformed elastically and to release the energy upon unloading.

Resilient: Will release substantially all of the energy when unloaded. Includes e.g. certain silicones, and thermoplastic elastomers.

Rigid structure or component: A structure or component that will not substantially change shape when subject to the loads typically encountered in use. An example of such a use may be setting up and maintaining a patient interface in sealing relationship with an entrance to a patient's airways, e.g. at a load of approximately 20 to 30 cmH2O pressure.

As an example, an I-beam may comprise a different bending stiffness (resistance to a bending load) in a first direction in comparison to a second, orthogonal direction. In another example, a structure or component may be floppy in a first direction and rigid in a second direction.

Stiffness (or rigidity) of a structure or component: The ability of the structure or component to resist deformation in response to an applied load. The load may be a force or a moment, e.g. compression, tension, bending or torsion. The structure or component may offer different resistances in different directions. The inverse of stiffness is flexibility.

Viscous: The ability of a material to resist flow.

Visco-elasticity: The ability of a material to display both elastic and viscous behaviour in deformation.

Yield: The situation when a material can no longer return back to its original geometry after deformation.

### 5.4 Structural Elements

Compression member: A structural element that resists compression forces.

Elbow: An elbow is an example of a structure that directs an axis of flow of air travelling therethrough to change direction through an angle. In one form, the angle may be approximately 90 degrees. In another form, the angle may be more, or less than 90 degrees. The elbow may have an approximately circular cross-section. In another form the elbow may have an oval or a rectangular cross-section. In certain forms an elbow may be rotatable with respect to a mating component, e.g. about 360 degrees. In certain forms an elbow may be removable from a mating component, e.g. via a snap connection. In certain forms, an elbow may be assembled to a mating component via a one-time snap during manufacture, but not removable by a patient.

Frame: Frame will be taken to mean a mask structure that bears the load of tension between two or more points of connection with a headgear. A mask frame may be a non-airtight load bearing structure in the mask. However, some forms of mask frame may also be air-tight.

Membrane: Membrane will be taken to mean a typically thin element that has, preferably, substantially no resistance to bending, but has resistance to being stretched.

Tie (noun): A structure designed to resist tension.

Thin structures:

Beams,

A beam may be relatively long in one dimension compared to the other two dimensions such that the smaller dimensions are comparatively thin compared to the long dimension

Membranes,

Relatively long in two dimensions, with one thin dimension. Readily deforms in response to bending forces. Resists being stretched, (might also resist compression).

### 5.5 Plates & Shells

These may be relatively long in two directions, with one thin dimension. They may have bending, tensile, and/or compressive stiffness.

### 5.6 Thick Structures: Solids

Seal: May be a noun form (“a seal”) which refers to a structure, or a verb form (“to seal”) which refers to the effect. Two elements may be constructed and/or arranged to ‘seal’ or to effect ‘sealing’ therebetween without requiring a separate ‘seal’ element per se.

Shell: A shell will be taken to mean a curved, relatively thin structure having bending, tensile and compressive stiffness. For example, a curved structural wall of a mask may be a shell. In some forms, a shell may be faceted. In some forms a shell may be airtight. In some forms a shell may not be airtight.

Stiffener: A stiffener will be taken to mean a structural component designed to increase the bending resistance of another component in at least one direction.

Strut: A strut will be taken to be a structural component designed to increase the compression resistance of another component in at least one direction.

Swivel (noun): A subassembly of components configured to rotate about a common axis, preferably independently, preferably under low torque. In one form, the swivel may be constructed to rotate through an angle of at least 360 degrees. In another form, the swivel may be constructed to rotate through an angle less than 360 degrees. When used in the context of an air delivery conduit, the sub-assembly of components preferably comprises a matched pair of cylindrical conduits. There may be little or no leak flow of air from the swivel in use.

### 5.7 Respiratory Cycle

Apnea: According to some definitions, an apnea is said to have occurred when flow falls below a predetermined threshold for a duration, e.g. 10 seconds. An obstructive apnea will be said to have occurred when, despite patient effort, some obstruction of the airway does not allow air to flow. A central apnea will be said to have occurred when an apnea is detected that is due to a reduction in breathing effort, or the absence of breathing effort, despite the airway being patent. A mixed apnea occurs when a reduction or absence of breathing effort coincides with an obstructed airway.

Breathing rate: The rate of spontaneous respiration of a patient, usually measured in breaths per minute.

Duty cycle: The ratio of inhalation time, Ti to total breath time, Ttot.

Effort (breathing): The work done by a spontaneously breathing person attempting to breathe.

Expiratory portion of a breathing cycle: The period from the start of expiratory flow to the start of inspiratory flow.

Flow limitation: Flow limitation will be taken to be the state of affairs in a patient's respiration where an increase in effort by the patient does not give rise to a corresponding increase in flow. Where flow limitation occurs during an inspiratory portion of the breathing cycle it may be described as inspiratory flow limitation. Where flow limitation occurs during an expiratory portion of the breathing cycle it may be described as expiratory flow limitation.

Types of flow limited inspiratory waveforms:
(i) Flattened: Having a rise followed by a relatively flat portion, followed by a fall.
(ii) M-shaped: Having two local peaks, one at the leading edge, and one at the trailing edge, and a relatively flat portion between the two peaks.
(iii) Chair-shaped: Having a single local peak, the peak being at the leading edge, followed by a relatively flat portion.
(iv) Reverse-chair shaped: Having a relatively flat portion followed by single local peak, the peak being at the trailing edge.

Hypopnea: According to some definitions, a hypopnea is taken to be a reduction in flow, but not a cessation of flow. In one form, a hypopnea may be said to have occurred when there is a reduction in flow below a threshold rate for a duration. A central hypopnea will be said to have occurred when a hypopnea is detected that is due to a reduction in breathing effort. In one form in adults, either of the following may be regarded as being hypopneas:
(i) a 30% reduction in patient breathing for at least 10 seconds plus an associated 4% desaturation; or
(ii) a reduction in patient breathing (but less than 50%) for at least 10 seconds, with an associated desaturation of at least 3% or an arousal.

Hyperpnea: An increase in flow to a level higher than normal.

Inspiratory portion of a breathing cycle: The period from the start of inspiratory flow to the start of expiratory flow will be taken to be the inspiratory portion of a breathing cycle.

Patency (airway): The degree of the airway being open, or the extent to which the airway is open. A patent airway is open. Airway patency may be quantified, for example with a value of one (1) being patent, and a value of zero (0), being closed (obstructed).

Positive End-Expiratory Pressure (PEEP): The pressure above atmosphere in the lungs that exists at the end of expiration.

Peak flow rate (Qpeak): The maximum value of flow rate during the inspiratory portion of the respiratory flow waveform.

Respiratory flow rate, patient airflow rate, respiratory airflow rate (Qr): These terms may be understood to refer to the RPT device's estimate of respiratory flow rate, as opposed to “true respiratory flow rate” or “true respiratory flow rate”, which is the actual respiratory flow rate experienced by the patient, usually expressed in litres per minute.

Tidal volume (Vt): The volume of air inhaled or exhaled during normal breathing, when extra effort is not applied. In principle the inspiratory volume Vi (the volume of air inhaled) is equal to the expiratory volume Ve (the volume of air exhaled), and therefore a single tidal volume Vt may be defined as equal to either quantity. In practice the tidal volume Vt is estimated as some combination, e.g. the mean, of the inspiratory volume Vi and the expiratory volume Ve.

Inhalation Time (Ti): The duration of the inspiratory portion of the respiratory flow rate waveform.

Exhalation Time (Te): The duration of the expiratory portion of the respiratory flow rate waveform.

Total Time (Ttot): The total duration between the start of one inspiratory portion of a respiratory flow rate waveform and the start of the following inspiratory portion of the respiratory flow rate waveform.

Typical recent ventilation: The value of ventilation around which recent values of ventilation Vent over some predetermined timescale tend to cluster, that is, a measure of the central tendency of the recent values of ventilation.

Upper airway obstruction (UAO): includes both partial and total upper airway obstruction. This may be associated with a state of flow limitation, in which the flow rate increases only slightly or may even decrease as the pressure difference across the upper airway increases (Starling resistor behaviour).

Ventilation (Vent): A measure of a rate of gas being exchanged by the patient's respiratory system. Measures of ventilation may include one or both of inspiratory and expiratory flow, per unit time. When expressed as a volume per minute, this quantity is often referred to as “minute ventilation”. Minute ventilation is sometimes given simply as a volume, understood to be the volume per minute.

### 5.8 Ventilation

Adaptive Servo-Ventilator (ASV): A servo-ventilator that has a changeable, rather than fixed target ventilation. The changeable target ventilation may be learned from some characteristic of the patient, for example, a respiratory characteristic of the patient.

Backup rate: A parameter of a ventilator that establishes the minimum breathing rate (typically in number of breaths per minute) that the ventilator will deliver to the patient, if not triggered by spontaneous respiratory effort.

Cycled: The termination of a ventilator's inspiratory phase. When a ventilator delivers a breath to a spontaneously breathing patient, at the end of the inspiratory portion of the breathing cycle, the ventilator is said to be cycled to stop delivering the breath.

Expiratory positive airway pressure (EPAP): a base pressure, to which a pressure varying within the breath is added to produce the desired interface pressure which the ventilator will attempt to achieve at a given time.

End expiratory pressure (EEP): Desired interface pressure which the ventilator will attempt to achieve at the end of the expiratory portion of the breath. If the pressure waveform template Π(Φ) is zero-valued at the end of expiration, i.e. Π(Φ)=0 when Φ=1, the EEP is equal to the EPAP.

Inspiratory positive airway pressure (IPAP): Maximum desired interface pressure which the ventilator will attempt to achieve during the inspiratory portion of the breath.

Pressure support: A number that is indicative of the increase in pressure during ventilator inspiration over that during ventilator expiration, and generally means the difference in pressure between the maximum value during inspiration and the base pressure (e.g., PS=IPAP−EPAP). In some contexts, pressure support means the difference which the ventilator aims to achieve, rather than what it actually achieves.

Servo-ventilator: A ventilator that measures patient ventilation, has a target ventilation, and which adjusts the level of pressure support to bring the patient ventilation towards the target ventilation.

Spontaneous/Timed (S/T): A mode of a ventilator or other device that attempts to detect the initiation of a breath of a spontaneously breathing patient. If however, the device is unable to detect a breath within a predetermined period of time, the device will automatically initiate delivery of the breath.

Swing: Equivalent term to pressure support.

Triggered: When a ventilator, or other respiratory therapy device such as an RPT device or portable oxygen concentrator, delivers a volume of breathable gas to a spontaneously breathing patient, it is said to be triggered to do so. Triggering usually takes place at or near the initiation of the respiratory portion of the breathing cycle by the patient's efforts.

### 5.9 Anatomy

### Anatomy of the Face

Ala: the external outer wall or “wing” of each nostril (plural: alar)

Alar angle: An angle formed between the ala of each nostril.

Alare: The most lateral point on the nasal ala.

Alar curvature (or alar crest) point: The most posterior point in the curved base line of each ala, found in the crease formed by the union of the ala with the cheek.

Auricle: The whole external visible part of the ear.

(nose) Bony framework: The bony framework of the nose comprises the nasal bones, the frontal process of the maxillae and the nasal part of the frontal bone.

(nose) Cartilaginous framework: The cartilaginous framework of the nose comprises the septal, lateral, major and minor cartilages.

Columella: the strip of skin that separates the nares and which runs from the pronasale to the upper lip.

Columella angle: The angle between the line drawn through the midpoint of the nostril aperture and a line drawn perpendicular to the Frankfort horizontal while intersecting subnasale.

Frankfort horizontal plane: A line extending from the most inferior point of the orbital margin to the left tragion. The tragion is the deepest point in the notch superior to the tragus of the auricle.

Glabella: Located on the soft tissue, the most prominent point in the midsagittal plane of the forehead.

Lateral nasal cartilage: A generally triangular plate of cartilage. Its superior margin is attached to the nasal bone and frontal process of the maxilla, and its inferior margin is connected to the greater alar cartilage.

Lip, lower (labrale inferius): The lip extending between the subnasale and the mouth.

Lip, upper (labrale superius): The lip extending between the mouth and the supramenton.

Greater alar cartilage: A plate of cartilage lying below the lateral nasal cartilage. It is curved around the anterior part of the naris. Its posterior end is connected to the frontal process of the maxilla by a tough fibrous membrane containing three or four minor cartilages of the ala.

Nares (Nostrils): Approximately ellipsoidal apertures forming the entrance to the nasal cavity. The singular form of nares is naris (nostril). The nares are separated by the nasal septum.

Naso-labial sulcus or Naso-labial fold: The skin fold or groove that runs from each side of the nose to the corners of the mouth, separating the cheeks from the upper lip.

Naso-labial angle: The angle between the columella and the upper lip, while intersecting subnasale.

Otobasion inferior: The lowest point of attachment of the auricle to the skin of the face.

Otobasion superior: The highest point of attachment of the auricle to the skin of the face.

Pronasale: the most protruded point or tip of the nose, which can be identified in lateral view of the rest of the portion of the head.

Philtrum: the midline groove that runs from lower border of the nasal septum to the top of the lip in the upper lip region.

Pogonion: Located on the soft tissue, the most anterior midpoint of the chin.

Ridge (nasal): The nasal ridge is the midline prominence of the nose, extending from the Sellion to the Pronasale.

Sagittal plane: A vertical plane that passes from anterior (front) to posterior (rear). The midsagittal plane is a sagittal plane that divides the body into right and left halves.

Sellion: Located on the soft tissue, the most concave point overlying the area of the frontonasal suture.

Septal cartilage (nasal): The nasal septal cartilage forms part of the septum and divides the front part of the nasal cavity.

Subalare: The point at the lower margin of the alar base, where the alar base joins with the skin of the superior (upper) lip.

Subnasal point: Located on the soft tissue, the point at which the columella merges with the upper lip in the midsagittal plane.

Supramenton: The point of greatest concavity in the midline of the lower lip between labrale inferius and soft tissue pogonion

### Anatomy of the Skull

Frontal bone: The frontal bone includes a large vertical portion, the squama frontalis, corresponding to the region known as the forehead.

Mandible: The mandible forms the lower jaw. The mental protuberance is the bony protuberance of the jaw that forms the chin.

Maxilla: The maxilla forms the upper jaw and is located above the mandible and below the orbits. The frontal process of the maxilla projects upwards by the side of the nose, and forms part of its lateral boundary.

Nasal bones: The nasal bones are two small oblong bones, varying in size and form in different individuals; they are placed side by side at the middle and upper part of the face, and form, by their junction, the “bridge” of the nose.

Nasion: The intersection of the frontal bone and the two nasal bones, a depressed area directly between the eyes and superior to the bridge of the nose.

Occipital bone: The occipital bone is situated at the back and lower part of the cranium. It includes an oval aperture, the foramen magnum, through which the cranial cavity communicates with the vertebral canal. The curved plate behind the foramen magnum is the squama occipitalis.

Orbit: The bony cavity in the skull to contain the eyeball.

Parietal bones: The parietal bones are the bones that, when joined together, form the roof and sides of the cranium.

Temporal bones: The temporal bones are situated on the bases and sides of the skull, and support that part of the face known as the temple.

Zygomatic bones: The face includes two zygomatic bones, located in the upper and lateral parts of the face and forming the prominence of the cheek.

### 5.10 Anatomy of the Respiratory System

Diaphragm: A sheet of muscle that extends across the bottom of the rib cage. The diaphragm separates the thoracic cavity, containing the heart, lungs and ribs, from the abdominal cavity. As the diaphragm contracts the volume of the thoracic cavity increases and air is drawn into the lungs.

Larynx: The larynx, or voice box houses the vocal folds and connects the inferior part of the pharynx (hypopharynx) with the trachea.

Lungs: The organs of respiration in humans. The conducting zone of the lungs contains the trachea, the bronchi, the bronchioles, and the terminal bronchioles. The respiratory zone contains the respiratory bronchioles, the alveolar ducts, and the alveoli.

Nasal cavity: The nasal cavity (or nasal fossa) is a large air filled space above and behind the nose in the middle of the face. The nasal cavity is divided in two by a vertical fin called the nasal septum. On the sides of the nasal cavity are three horizontal outgrowths called nasal conchae (singular “concha”) or turbinates. To the front of the nasal cavity is the nose, while the back blends, via the choanae, into the nasopharynx.

Pharynx: The part of the throat situated immediately inferior to (below) the nasal cavity, and superior to the oesophagus and larynx. The pharynx is conventionally divided into three sections: the nasopharynx (epipharynx) (the nasal part of the pharynx), the oropharynx (mesopharynx) (the oral part of the pharynx), and the laryngopharynx (hypopharynx).

### 5.11 Patient Interface

Anti-asphyxia valve (AAV): The component or sub-assembly of a mask system that, by opening to atmosphere in a failsafe manner, reduces the risk of excessive CO2 rebreathing by a patient.

Headgear: Headgear will be taken to mean a form of positioning and stabilising structure designed to hold a device, e.g., a mask, on a head.

Plenum chamber: a mask plenum chamber will be taken to mean a portion of a patient interface having walls at least partially enclosing a volume of space, the volume having air therein pressurised above atmospheric pressure in use. A shell may form part of the walls of a mask plenum chamber.

Seal: May be a noun form (“a seal”) which refers to a structure, or a verb form (“to seal”) which refers to the effect. Two elements may be constructed and/or arranged to ‘seal’ or to effect ‘sealing’ therebetween without requiring a separate ‘seal’ element per se.

Vent: (noun): A structure that allows a flow of air from an interior of the mask, or conduit, to ambient air for clinically effective washout of exhaled gases. For example, a clinically effective washout may involve a flow rate of about 10 litres per minute to about 100 litres per minute, depending on the mask design and treatment pressure.
a. Shape of structures
b. Products in accordance with the present technology may comprise one or more three-dimensional mechanical structures, for example a mask cushion or an impeller. The three-dimensional structures may be bounded by two-dimensional surfaces. These surfaces may be distinguished using a label to describe an associated surface orientation, location, function, or some other characteristic. For example a structure may comprise one or more of an anterior surface, a posterior surface, an interior surface and an exterior surface. In another example, a seal-forming structure may comprise a face-contacting (e.g. outer) surface, and a separate non-face-contacting (e.g. underside or inner) surface. In another example, a structure may comprise a first surface and a second surface.
c. To facilitate describing the shape of the three-dimensional structures and the surfaces, we first consider a cross-section through a surface of the structure at a point, p. See FIG. 3B to FIG. 3F, which illustrate examples of cross-sections at point p on a surface, and the resulting plane curves. FIGS. 3B to 3F also illustrate an outward normal vector at p. The outward normal vector at p points away from the surface. In some examples we describe the surface from the point of view of an imaginary small person standing upright on the surface.
d. Curvature in one dimension
e. The curvature of a plane curve at p may be described as having a sign (e.g. positive, negative) and a magnitude (e.g. 1/radius of a circle that just touches the curve at p).
f. Positive curvature: If the curve at p turns towards the outward normal, the curvature at that point will be taken to be positive (if the imaginary small person leaves the point p they must walk uphill). See FIG. 3B (relatively large positive curvature compared to FIG. 3C) and FIG. 3C (relatively small positive curvature compared to FIG. 3B). Such curves are often referred to as concave.
g. Zero curvature: If the curve at p is a straight line, the curvature will be taken to be zero (if the imaginary small person leaves the point p, they can walk on a level, neither up nor down). See FIG. 3D.
h. Negative curvature: If the curve at p turns away from the outward normal, the curvature in that direction at that point will be taken to be negative (if the imaginary small person leaves the point p they must walk downhill). See FIG. 3E (relatively small negative curvature compared to FIG. 3F) and FIG. 3F (relatively large negative curvature compared to FIG. 3E). Such curves are often referred to as convex.
i. Curvature of two dimensional surfaces
j. A description of the shape at a given point on a two-dimensional surface in accordance with the present technology may include multiple normal cross-sections. The multiple cross-sections may cut the surface in a plane that includes the outward normal (a “normal plane”), and each cross-section may be taken in a different direction. Each cross-section results in a plane curve with a corresponding curvature. The different curvatures at that point may have the same sign, or a different sign. Each of the curvatures at that point has a magnitude, e.g. relatively small. The plane curves in FIGS. 3B to 3F could be examples of such multiple cross-sections at a particular point.
k. Principal curvatures and directions: The directions of the normal planes where the curvature of the curve takes its maximum and minimum values are called the principal directions. In the examples of FIG. 3B to FIG. 3F, the maximum curvature occurs in FIG. 3B, and the minimum occurs in FIG. 3F, hence FIG. 3B and FIG. 3F are cross sections in the principal directions. The principal curvatures at p are the curvatures in the principal directions.
l. Region of a surface: A connected set of points on a surface. The set of points in a region may have similar characteristics, e.g. curvatures or signs.
m. Saddle region: A region where at each point, the principal curvatures have opposite signs, that is, one is positive, and the other is negative (depending on the direction to which the imaginary person turns, they may walk uphill or downhill).
n. Dome region: A region where at each point the principal curvatures have the same sign, e.g. both positive (a “concave dome”) or both negative (a “convex dome”).
o. Cylindrical region: A region where one principal curvature is zero (or, for example, zero within manufacturing tolerances) and the other principal curvature is non-zero.
p. Planar region: A region of a surface where both of the principal curvatures are zero (or, for example, zero within manufacturing tolerances).
q. Edge of a surface: A boundary or limit of a surface or region.
r. Path: In certain forms of the present technology, ‘path’ will be taken to mean a path in the mathematical—topological sense, e.g. a continuous space curve from f(0) to f(1) on a surface. In certain forms of the present technology, a ‘path’ may be described as a route or course, including e.g. a set of points on a surface. (The path for the imaginary person is where they walk on the surface, and is analogous to a garden path).
s. Path length: In certain forms of the present technology, ‘path length’ will be taken to mean the distance along the surface from f(0) to f(1), that is, the distance along the path on the surface. There may be more than one path between two points on a surface and such paths may have different path lengths. (The path length for the imaginary person would be the distance they have to walk on the surface along the path).
t. Straight-line distance: The straight-line distance is the distance between two points on a surface, but without regard to the surface. On planar regions, there would be a path on the surface having the same path length as the straight-line distance between two points on the surface. On non-planar surfaces, there may be no paths having the same path length as the straight-line distance between two points. (For the imaginary person, the straight-line distance would correspond to the distance ‘as the crow flies’.)
u. Space curves
v. Space curves: Unlike a plane curve, a space curve does not necessarily lie in any particular plane. A space curve may be closed, that is, having no endpoints. A space curve may be considered to be a one-dimensional piece of three-dimensional space. An imaginary person walking on a strand of the DNA helix walks along a space curve. A typical human left ear comprises a helix, which is a left-hand helix, see FIG. 3Q. A typical human right ear comprises a helix, which is a right-hand helix, see FIG. 3R. FIG. 3S shows a right-hand helix. The edge of a structure, e.g. the edge of a membrane or impeller, may follow a space curve. In general, a space curve may be described by a curvature and a torsion at each point on the space curve. Torsion is a measure of how the curve turns out of a plane. Torsion has a sign and a magnitude. The torsion at a point on a space curve may be characterised with reference to the tangent, normal and binormal vectors at that point.
w. Tangent unit vector (or unit tangent vector): For each point on a curve, a vector at the point specifies a direction from that point, as well as a magnitude. A tangent unit vector is a unit vector pointing in the same direction as the curve at that point. If an imaginary person were flying along the curve and fell off her vehicle at a particular point, the direction of the tangent vector is the direction she would be travelling.
x. Unit normal vector: As the imaginary person moves along the curve, this tangent vector itself changes. The unit vector pointing in the same direction that the tangent vector is changing is called the unit principal normal vector. It is perpendicular to the tangent vector.
y. Binormal unit vector: The binormal unit vector is perpendicular to both the tangent vector and the principal normal vector. Its direction may be determined by a right-hand rule (see e.g. FIG. 3P), or alternatively by a left-hand rule (FIG. 3O).
z. Osculating plane: The plane containing the unit tangent vector and the unit principal normal vector. See FIGS. 3O and 3P.
aa. Torsion of a space curve: The torsion at a point of a space curve is the magnitude of the rate of change of the binormal unit vector at that point. It measures how much the curve deviates from the osculating plane. A space curve which lies in a plane has zero torsion. A space curve which deviates a relatively small amount from the osculating plane will have a relatively small magnitude of torsion (e.g. a gently sloping helical path). A space curve which deviates a relatively large amount from the osculating plane will have a relatively large magnitude of torsion (e.g. a steeply sloping helical path). With reference to FIG. 3S, since T2>T1, the magnitude of the torsion near the top coils of the helix of FIG. 3S is greater than the magnitude of the torsion of the bottom coils of the helix of FIG. 3S
bb. With reference to the right-hand rule of FIG. 3P, a space curve turning towards the direction of the right-hand binormal may be considered as having a right-hand positive torsion (e.g. a right-hand helix as shown in FIG. 3S). A space curve turning away from the direction of the right-hand binormal may be considered as having a right-hand negative torsion (e.g. a left-hand helix).
cc. Equivalently, and with reference to a left-hand rule (see FIG. 3O), a space curve turning towards the direction of the left-hand binormal may be considered as having a left-hand positive torsion (e.g. a left-hand helix). Hence left-hand positive is equivalent to right-hand negative. See FIG. 3T.
dd. Holes
ee. A surface may have a one-dimensional hole, e.g. a hole bounded by a plane curve or by a space curve. Thin structures (e.g. a membrane) with a hole, may be described as having a one-dimensional hole. See for example the one dimensional hole in the surface of structure shown in FIG. 3I, bounded by a plane curve.
ff. A structure may have a two-dimensional hole, e.g. a hole bounded by a surface. For example, an inflatable tyre has a two dimensional hole bounded by the interior surface of the tyre. In another example, a bladder with a cavity for air or gel could have a two-dimensional hole. See for example the cushion of FIG. 3L and the example cross-sections therethrough in FIG. 3M and FIG. 3N, with the interior surface bounding a two dimensional hole indicated. In a yet another example, a conduit may comprise a one-dimension hole (e.g. at its entrance or at its exit), and a two-dimension hole bounded by the inside surface of the conduit. See also the two dimensional hole through the structure shown in FIG. 3K, bounded by a surface as shown.

### 6 OTHER REMARKS

A portion of the disclosure of this patent document contains material which is subject to copyright protection. The copyright owner has no objection to the facsimile reproduction by anyone of the patent document or the patent disclosure, as it appears in Patent Office patent files or records, but otherwise reserves all copyright rights whatsoever.

Unless the context clearly dictates otherwise and where a range of values is provided, it is understood that each intervening value, to the tenth of the unit of the lower limit, between the upper and lower limit of that range, and any other stated or intervening value in that stated range is encompassed within the technology. The upper and lower limits of these intervening ranges, which may be independently included in the intervening ranges, are also encompassed within the technology, subject to any specifically excluded limit in the stated range. Where the stated range includes one or both of the limits, ranges excluding either or both of those included limits are also included in the technology.

Furthermore, where a value or values are stated herein as being implemented as part of the technology, it is understood that such values may be approximated, unless otherwise stated, and such values may be utilized to any suitable significant digit to the extent that a practical technical implementation may permit or require it.

Furthermore, “approximately”, “substantially”, “about”, or any similar term used herein means +/−5-10% of the recited value.

Unless defined otherwise, all technical and scientific terms used herein have the same meaning as commonly understood by one of ordinary skill in the art to which this technology belongs. Although any methods and materials similar or equivalent to those described herein can also be used in the practice or testing of the present technology, a limited number of the exemplary methods and materials are described herein.

When a particular material is identified as being used to construct a component, obvious alternative materials with similar properties may be used as a substitute. Furthermore, unless specified to the contrary, any and all components herein described are understood to be capable of being manufactured and, as such, may be manufactured together or separately.

It must be noted that as used herein and in the appended claims, the singular forms “a”, “an”, and “the” include their plural equivalents, unless the context clearly dictates otherwise.

All publications mentioned herein are incorporated herein by reference in their entirety to disclose and describe the methods and/or materials which are the subject of those publications. The publications discussed herein are provided solely for their disclosure prior to the filing date of the present application. Nothing herein is to be construed as an admission that the present technology is not entitled to antedate such publication by virtue of prior invention. Further, the dates of publication provided may be different from the actual publication dates, which may need to be independently confirmed.

The terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

The subject headings used in the detailed description are included only for the ease of reference of the reader and should not be used to limit the subject matter found throughout the disclosure or the claims. The subject headings should not be used in construing the scope of the claims or the claim limitations.

Although the technology herein has been described with reference to particular examples, it is to be understood that these examples are merely illustrative of the principles and applications of the technology. In some instances, the terminology and symbols may imply specific details that are not required to practice the technology. For example, although the terms “first” and “second” may be used, unless otherwise specified, they are not intended to indicate any order but may be utilised to distinguish between distinct elements. Furthermore, although process steps in the methodologies may be described or illustrated in an order, such an ordering is not required. Those skilled in the art will recognize that such ordering may be modified and/or aspects thereof may be conducted concurrently or even synchronously.

It is therefore to be understood that numerous modifications may be made to the illustrative examples and that other arrangements may be devised without departing from the spirit and scope of the technology.

1. A patient interface comprising:
a plenum chamber pressurisable to a therapeutic pressure of at least 6 cm H2O above ambient air pressure, said plenum chamber including a plenum chamber inlet port sized and structured to receive a flow of air at the therapeutic pressure for breathing by a patient,
a seal-forming structure constructed and arranged to form a seal with a region of the patient's face surrounding an entrance to the patient's airways, said seal-forming structure having a hole therein such that the flow of air at said therapeutic pressure is delivered to at least an entrance to the patient's nares, the seal-forming structure constructed and arranged to maintain said therapeutic pressure in the plenum chamber throughout the patient's respiratory cycle in use; and
a vent structure to allow a continuous flow of gases exhaled by the patient from an interior of the plenum chamber to ambient, said vent structure being sized and shaped to maintain the therapeutic pressure in the plenum chamber in use;
wherein the patient interface comprises a heat and moisture exchanger (HME) for heating and/or humidifying the flow of air for breathing by the patient using heat and/or moisture recovered from the flow of gases exhaled by the patient, the HME comprising a heat and moisture exchange component and a thermally conductive member to facilitate heat transfer within the HME, the thermally conductive member in contact with, or incorporated within, the heat and moisture exchange component.

2. The patient interface of claim 1, wherein the HME is located in or partially in the plenum chamber and the thermally conductive member or portion thereof is located inside the plenum chamber between the patient's face and the heat and moisture exchange component.

3. The patient interface of claim 1, wherein the patient interface includes a tube connected to the plenum chamber inlet port, the HME connected to the tube at an opposing tube end distal from the plenum chamber.

4. The patient interface of claim 3, wherein the thermally conductive member of the HME is located between the plenum chamber and the heat and moisture exchange component of the HME.

5. The patient interface of claim 1, wherein the thermally conductive member is in the form of one or more of; a metal sheet, a foil sheet, mesh, perforated metal sheeting, wire or plate.

6. The patient interface of claim 1, wherein the thermally conductive member is formed from a metal selected from silver, copper, gold, steel, aluminium nitride, silicon carbide, aluminium, tungsten, graphite or zinc.

7. The patient interface of claim 1, wherein the HME comprises a covering structure, flange, surround casing or retaining structure, the covering structure formed from, partially formed from, or comprising the thermally conductive member.

8. The patient interface of claim 1, wherein the thermally conductive member is a covering structure shaped to at least partially receive or be received by at least a portion of the heat and moisture exchange component.

9. The patient interface of claim 1, wherein the heat and moisture exchange component is impregnated and/or coated with the thermally conductive member.

10. The patient interface of claim 1, wherein the HME is shaped such that at least a portion of the thermally conductive member is exposed to ambient air.

11. The patient interface of claim 10, wherein the HME comprises a projection structure providing thermal connection between the thermally conductive member and ambient air.

12. A heat and moisture exchanger (HME) for heating and/or humidifying the flow of air for breathing by a patient using heat and/or moisture recovered from a flow of gases exhaled by the patient, the HME comprising a heat and moisture exchange component and a thermally conductive member to facilitate heat transfer within the HME, the thermally conductive member in contact with, or incorporated within, the heat and moisture exchange component, for incorporation into a patient interface, the patient interface comprising;
key things that we want to stop a competitor from doing a plenum chamber pressurisable to a therapeutic pressure of at least 6 cm H2O above ambient air pressure, said plenum chamber including a plenum chamber inlet port sized and structured to receive a flow of air at the therapeutic pressure for breathing by a patient,
a seal-forming structure constructed and arranged to form a seal with a region of the patient's face surrounding an entrance to the patient's airways, said seal-forming structure having a hole therein such that the flow of air at said therapeutic pressure is delivered to at least an entrance to the patient's nares, the seal-forming structure constructed and arranged to maintain said therapeutic pressure in the plenum chamber throughout the patient's respiratory cycle in use; and
a vent structure to allow a continuous flow of gases exhaled by the patient from an interior of the plenum chamber to ambient, said vent structure being sized and shaped to maintain the therapeutic pressure in the plenum chamber in use.
wherein the HME is removably connected directly or indirectly to the plenum chamber.

13. The heat and moisture exchanger (HME) of claim 12, wherein when connected to the patient interface, the thermally conductive member of the HME or portion thereof is located on or in the plenum chamber between the patient's face and the heat and moisture exchange component.

14. The heat and moisture exchanger (HME) of claim 13 wherein the patient interface includes a tube connected to the plenum chamber inlet port, the HME connected to the tube at an opposing tube end distal from the plenum chamber.

15. The heat and moisture exchanger (HME) of claim 14, wherein the thermally conductive member is in the form of or comprises one or more of; a metal sheet, a foil sheet, mesh, perforated metal sheeting, wire or plate.

16. The heat and moisture exchanger (HME) of claim 12, wherein the HME comprises a covering structure, flange, surround, casing or retaining structure formed from, partially formed from, or comprising the thermally conductive member.

17. The heat and moisture exchanger (HME) of claim 12, wherein the thermally conductive member is a covering structure shaped to at least partially receive or be received by at least a portion of the heat and moisture exchange component.

18. The heat and moisture exchanger (HME) of claim 12, wherein the HME is shaped such that at least a portion of the thermally conductive member is exposed to ambient air.

19. The heat and moisture exchanger (HME) of claim 12, wherein the HME comprises a projection structure providing thermal connection between the thermally conductive member and ambient air.

20. The heat and moisture exchanger (HME) of claim 12, wherein the thermally conductive member is removably connected to the heat and moisture exchange component and/or a component of the patient interface.

21. A heat and moisture exchanger (HME) for heating and/or humidifying the flow of air for breathing by a patient using heat and/or moisture recovered from a flow of gases exhaled by the patient, the HME comprising a heat and moisture exchange component and a thermally conductive member to facilitate heat transfer within the HME, the thermally conductive member being in contact with, or incorporated within, the heat and moisture exchange component, for incorporation into a patient interface having a plenum chamber pressurisable to a therapeutic pressure of at least 6 cm H2O above ambient air pressure, wherein the HME is configured to be removably connected directly or indirectly to the plenum chamber.
